(19) United States
(12) Patent Application Publication
(10) Pub. No.: US 2025/0090079 A1
(43) Pub. Date: Mar. 20, 2025

(54) DUAL-PURPOSING A SENSING COMPONENT ON A WEARABLE DEVICE TO ALSO PERFORM A HAPTIC-RENDERING FUNCTION, INCLUDING USING PREDETERMINED HAPTIC PRECURSORS TO TARGET BIOPHYSICAL AREAS OF A USER, AND SYSTEMS AND METHODS OF USE THEREOF

(71) Applicant: Meta Platforms Technologies, LLC, Menlo Park, CA (US)

(72) Inventors: Brendan Patrick Flynn, Redmond, WA (US); Benjamin Stephens-Fripp, Redmond, WA (US); Cameron Miner, Brentwood, NH (US)

(21) Appl. No.: 18/752,511

(22) Filed: Jun. 24, 2024

Related U.S. Application Data
(60) Provisional application No. 63/583,221, filed on Sep. 15, 2023.

(51) Int. Cl. A61B 5/383; A61B 5/00; A61B 5/395; G02B 27/01; G06F 3/01

(52) U.S. Cl. CPC A61B 5/383; A61B 5/395; A61B 5/681; G02B 27/017; G06F 3/015; G02B 2027/0178

(57) ABSTRACT

Methods, systems, and devices for sensing biometric signals and sending haptic precursors to effectuate a haptic sensation is disclosed. Utilizing a component, for example a sensing component or electrode, on a wearable device, a biometric signal of a user can be sensed. One or more haptic precursors can also be sent by the component to one or more targeted biophysical areas of the user. The one or more haptic precursors can cause the user to sense one or more haptic sensations at the targeted biophysical areas.

### RELATED APPLICATION

This application claims priority to U.S. Provisional Application Ser. No. 63/583,221, filed Sep. 15, 2023, entitled “Dual-Purposing a Sensing Component on a Wearable Device to Also Perform a Haptic-Rendering Function, Including Using Predetermined Haptic Precursors to Target Biophysical Areas of a User, and Systems and Methods of Use Thereof,” which is hereby incorporated herein by reference in its entirety.

### TECHNICAL FIELD

This application relates generally to wearable devices and using components, for example sensing components, on wearable devices for sending haptic precursors to a user such that the user experiences a haptic sensation. At least one haptic precursor can be sent to a particular targeted biophysical area of the user to effectuate a particular haptic sensation. Additional or varied haptic precursors with varied precursor characteristics can also be applied to effectuate varying haptic sensations. These kinds of wearable devices and the methods related thereto can be used in the context of artificial reality and in connection with wearable headsets.

### BACKGROUND

Techniques for providing haptic sensations do exist, but they come in the form of very bulky and cumbersome wearable devices that limit the user's freedom of movement. In addition, employing these techniques while a user is immersed in an artificial-reality environment can detract from immersion within the artificial-reality environment and/or can interrupt a user's sustained interaction with the artificial-reality environment if the haptic sensation that is provided is too generalized and not targeted to a smaller area of the user's body. Further, these devices typically allow for providing haptic sensations that are generalized to larger areas of the user's body and do not specifically target individual muscle groups to provide finely focused haptic sensations. Accordingly, there is a need for a user-friendly, finely focused, and more realistic method of effectuating sensations and movement of a user at least so as to not detract from the immersion within the artificial reality.

As such, there is a need to address one or more of the above-identified challenges. A brief summary of solutions to the issues noted above are described below.

### SUMMARY

The methods, systems, and devices described herein allow users wearing wearable devices (e.g., smart watches, smart necklaces, smart headbands, smart armbands, and other articles of clothing made “smart” through the incorporation of electronic components) to receive haptic feedback that targets specific areas of the user's body (e.g., target specific biophysical areas, such as one specific muscle group), which can facilitate engaging with an artificial environment in an immersive and interactive manner, which is one use case and others are also contemplated such as providing targeted haptic feedback while using a gaming platform or while interacting directly with a display of the wearable device, as a few non-limiting example use cases. In some embodiments, a component is dual-purposed, for example an electrode or other sensing component, on a wearable device, which electrode is used for sensing a biometric signal of a user. By dual-purposing the component, in addition to performing its sensing function, the component can also be used to send one or more haptic stimulations to target one or more biophysical areas of the user. The one or more haptic stimulations can cause the user to sense one or more haptic sensations at a targeted biophysical area.

One example of a method of repurposing (for circumstances in which the sensing component is used to perform the haptic-rendering function while not performing its normal sensing function, the sensing component can be understood to have been temporarily repurposed, and thus the descriptions herein occasionally refer to “repurposing” while also referring to “dual-purposing” of sensing components as the components are able to perform multiple functions (e.g., sensing and rendering)) a sensing component to perform a haptic-rendering function is described herein. This example method includes, at a first point in time, using a sensing component of a wearable electronic device to sense a biometric signal (e.g., a neuromuscular signal, such as an electromyography signal) of a user. The example method further includes, at a second point in time that is distinct from the first point in time, instructing the sensing component to send a predetermined haptic precursor to a targeted biophysical area of the user, such that the user is caused to perceive a haptic sensation after the predetermined haptic precursor is received at the targeted biophysical area of the user.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIGS. 1A-1G illustrate an example system configured for sending at least one haptic precursor to a targeted biophysical area, in accordance with some embodiments.

FIG. 2 shows an example method flow chart for a method of sending at least one haptic precursor, in accordance with some embodiments.

FIGS. 3A-3E illustrate example wrist-wearable devices, in accordance with some embodiments.

FIGS. 4A-4B illustrate example artificial-reality systems, in accordance with some embodiments.

FIGS. 5A-5B are block diagrams illustrating an example artificial-reality system, in accordance with some embodiments.

In accordance with common practice, the various features illustrated in the drawings may not be drawn to scale. Accordingly, the dimensions of the various features can be arbitrarily expanded or reduced for clarity. In addition, some of the drawings do not necessarily depict all of the components of a given system, method, or device. Finally, in accordance with common practice, like reference numerals are used to denote like features throughout the specification and figures.

### DETAILED DESCRIPTION

Numerous details are described herein to provide a thorough understanding of the example embodiments illustrated in the accompanying drawings. However, some embodiments can be practiced without many of the specific details. Furthermore, well-known processes, components, and materials have not necessarily been described in exhaustive detail so as to avoid obscuring pertinent aspects of the embodiments described herein.

Having summarized example solutions and briefly introduced each of the drawings, provided next is a detailed description of various techniques for repurposing or dual-purposing a sensing component to perform a haptic-rendering function (e.g., an electrode on a wrist-wearable device that is used for sensing of biopotential signals (e.g., EMG signals) that can temporarily cease sensing the biopotential signals and instead be repurposed to send a haptic precursor to a targeted biophysical area of a user, such that the user is caused to feel a haptic sensation at the targeted biophysical area of the user). Multiple biophysical areas, which can be specific muscle groups or tendons (among other examples), can be targeted in a substantially simultaneous fashion by repurposing more than one sensing component to target each respective biophysical area of the multiple biophysical areas, thereby resulting in a coordinated haptic effect that is caused to be felt by the user at the multiple biophysical areas.

While the use of repurposed or dual-purposed sensing components can be in conjunction with a wearable device on its own (e.g., while a user interacts with a display of the wearable device), in other examples, the techniques can be implemented in conjunction with various types or embodiments of artificial-reality systems. Artificial reality, as described herein, is any superimposed functionality and/or sensory-detectable presentation provided by an artificial-reality system within a user's physical surroundings. Such artificial realities can include and/or represent virtual reality (VR), augmented reality (AR), mixed artificial reality (MAR), or some combination of these and/or variation of one of these. For example, a user can perform a swiping in-air hand gesture to cause a song to be skipped by a song-providing application programming interface (API) providing playback at, for example, a home speaker. In some embodiments of an AR system, ambient light (e.g., a live feed of the surrounding environment that a user would normally see) can be passed through a display element of a respective head-wearable device presenting aspects of the AR system. In some embodiments, ambient light can be passed through respective aspects of the AR system. For example, a visual user interface element (e.g., a notification user interface element) can be presented at the head-wearable device, and an amount of ambient light (e.g., 15-50% of the ambient light) can be passed through the user interface element, such that the user can distinguish at least a portion of the physical environment over which the user interface element is being displayed.

Artificial-reality content can include completely generated content or generated content combined with captured (e.g., real-world) content. The artificial-reality content can include video, audio, haptic events, or some combination thereof, any of which can be presented in a single channel or in multiple channels (such as stereo video that produces a three-dimensional effect to a viewer). Additionally, in some embodiments, artificial reality can also be associated with applications, products, accessories, services, or some combination thereof, which are used, for example, to create content in an artificial reality and/or are otherwise used in (e.g., to perform activities in) an artificial reality.

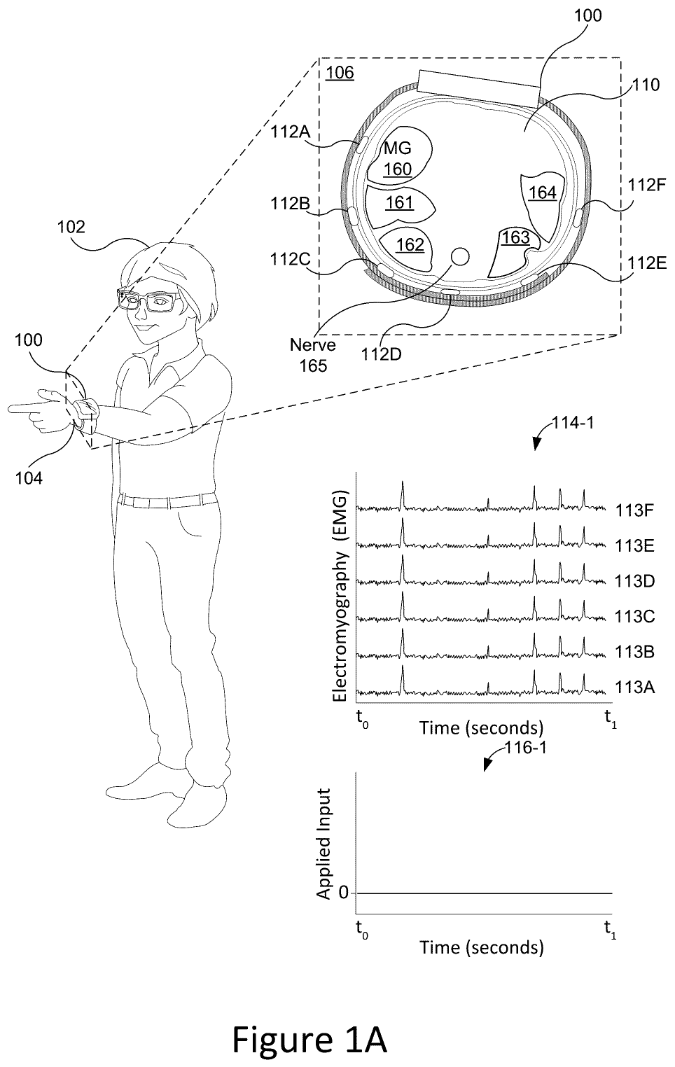

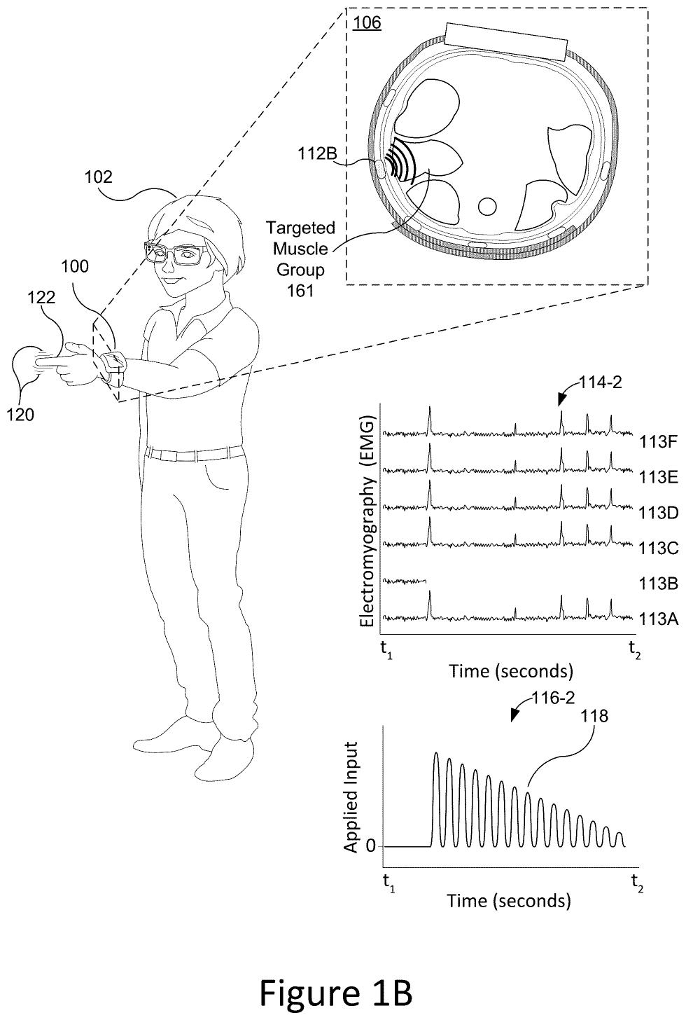

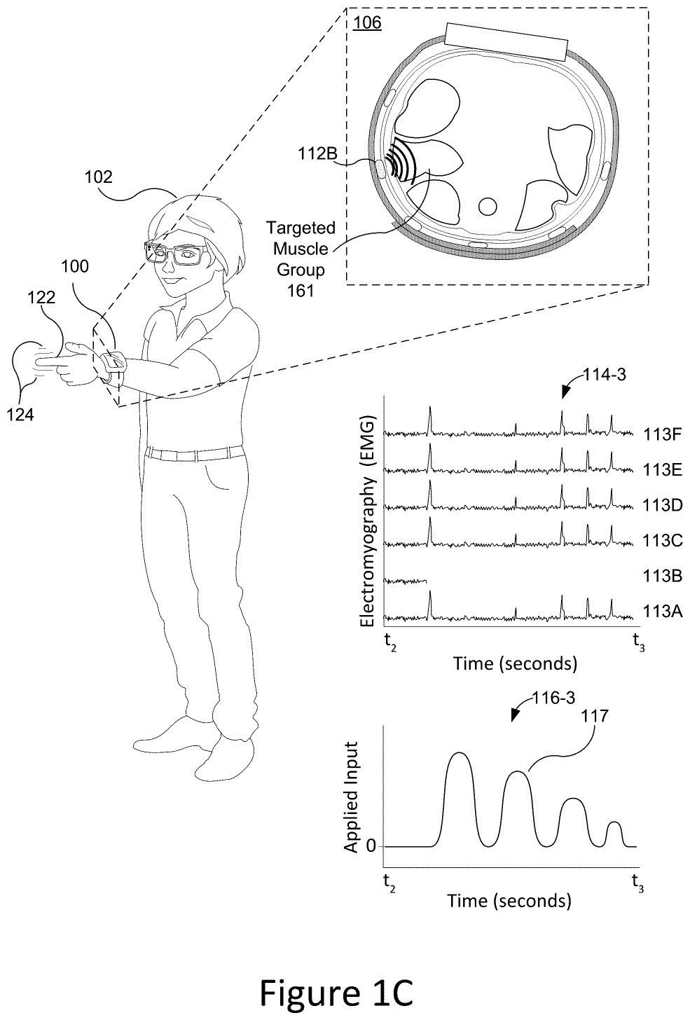

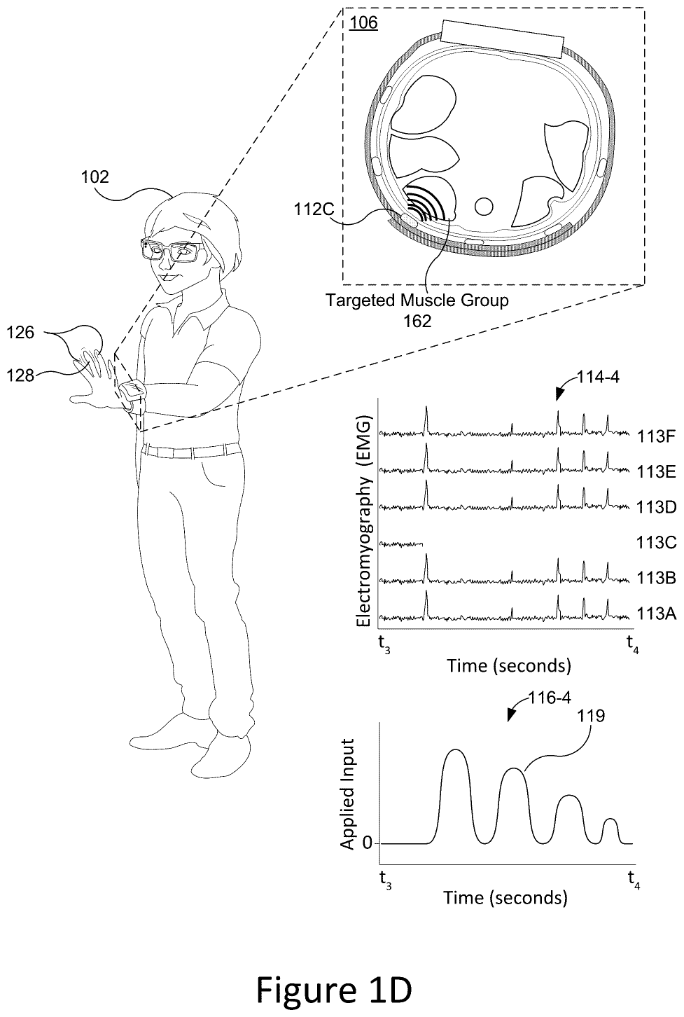

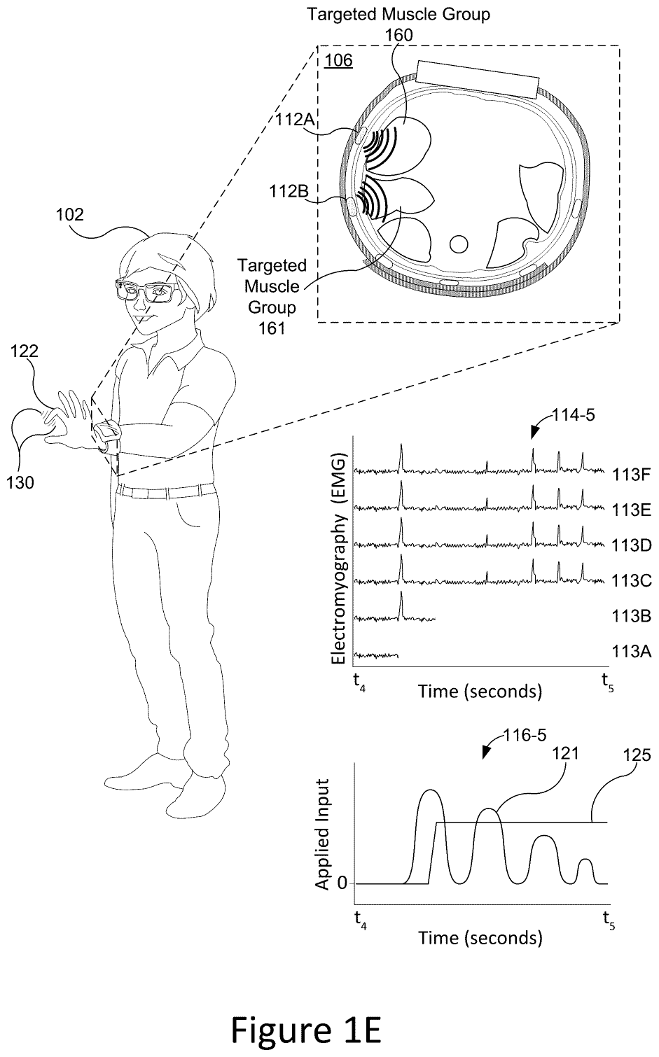

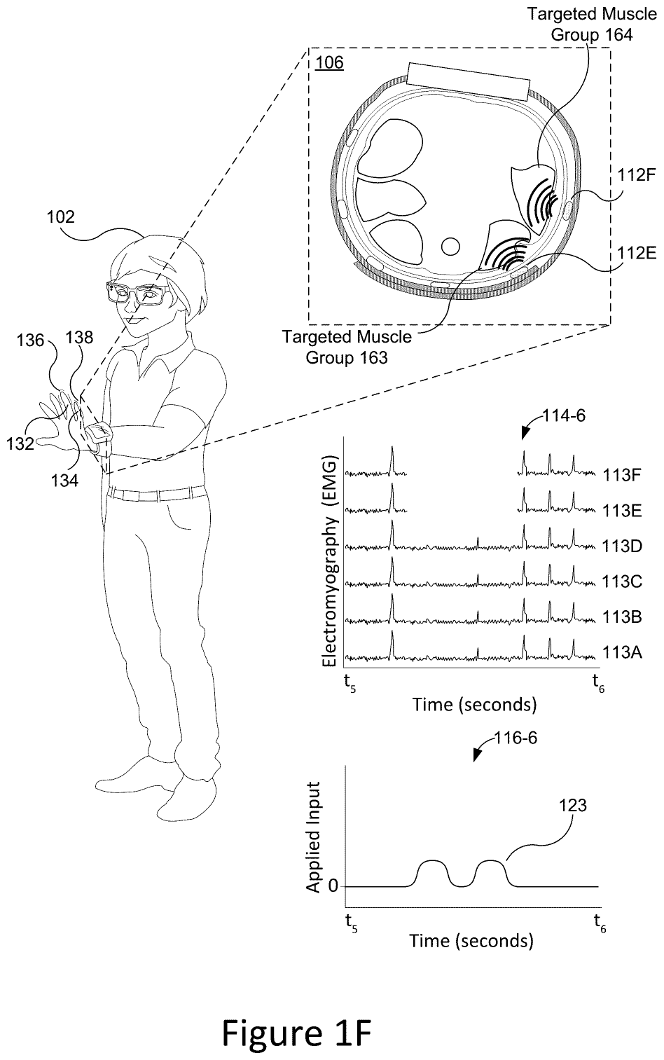

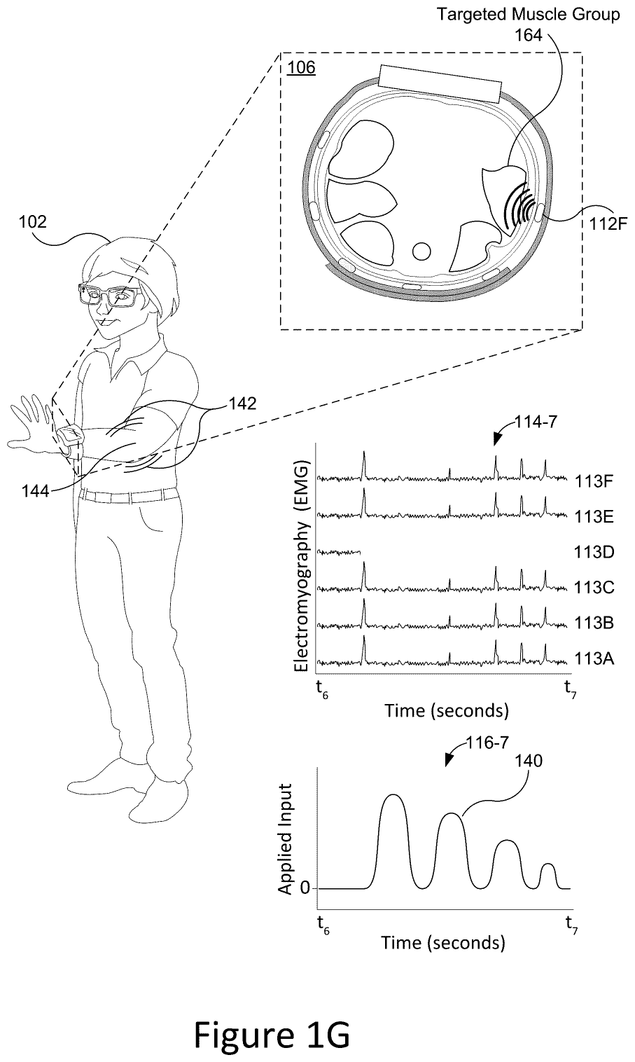

FIG. 1A illustrates a user 102 wearing a wearable device 100 that includes a plurality of sensing components that can also perform a haptic rendering function, in accordance with some embodiments. FIG. 1A shows an example wearable device 100 being worn by a user 102 around the user's wrist 104. A cutaway 106 is shown that includes a cutaway of the wearable device 100 and a cutaway of the user's wrist 110 (showing biophysical areas of the user's wrist 104, including a plurality of muscle groups (MG) 160-164 and nerve 165). Other biophysical areas can also be targeted, for example muscle groups located in another location in the wrist or forearm, one or more tendons, or one or more nerves (e.g., median nerve, ulnar nerve, radial nerve, etc.). The cutaway of the wearable device 100 shows sensing components 112A-112F (e.g., neuromuscular signal sensors, such as electromyography sensors) being placed around the band(s) of the cutaway of the wearable device 100. The number and position of the sensing components can be varied (e.g., the number of sensing components can be increased or decreased, the distribution can be varied and organized based locations of biophysical areas to be targeted, etc.). The sensing components 112A-112F are configured to both receive biophysical data (e.g., biopotential signals, including those used to determine a heart rate, electromyography (EMG) data, etc.) and apply a haptic precursor (e.g., electrical current having an adjustable current, amplitude, frequency, and phase) directed toward one or more biophysical areas of the user to cause a haptic feedback sensation sensed by the user 102. FIG. 1A also shows a chart 114-1 that indicates the biometric signals being recorded over a first time period measured in seconds, i.e., t₀-t₁, of each of the sensing components 112A-112F. Chart 114-1 includes six separate biometric signal lines 113A-113F that correspond respectively to the signals sensed by sensing components 112A-112F (i.e., biometric signal line 113A corresponds to the biometric signal sensed by sensing component 112A, biometric signal line 113B corresponds to the biometric signal sensed by sensing component 112B, and so on). In some embodiments, the biometric signals of each sensing component are sensed continuously and simultaneously. In some embodiments, the biometric signals are only sensed and recorded by certain sensing components. A chart 116-1 is also shown in FIG. 1A that indicates measurements of a haptic precursor being applied to a targeted biophysical area of the user over the first time period measured in seconds, i.e., t₀-t₁. However, FIG. 1A presently shows that no haptic precursor is being applied across the first time period. While FIG. 1A and subsequent figures show the wearable device being worn around the user's wrist, the wearable device can be configured to be worn on other portions of the body to achieve similar functionalities (e.g., worn around the forearm, upper arm, legs, ankles, head, neck, chest, etc.), and sensing components of such wearable devices worn on other portions of the body can also be used to perform the haptic-rendering functions discussed herein.

FIG. 1B illustrates a second point in time of the user 102 wearing the wearable device 100 where the sensing component 112B directs a predetermined haptic precursor (e.g., the haptic precursor can be stored in a memory of the wearable device and retrieved when the wearable device identifies an appropriate time to direct a particular predetermined haptic precursor toward an intended biophysical area of the user) toward a targeted biophysical area of the user, such that the user is caused to perceive a haptic sensation after the haptic precursor is received at the targeted biophysical area of the user. FIG. 1B illustrates the predetermined haptic precursor being directed toward targeted muscle group 161. As discussed elsewhere in this specification, the targeted biophysical area of the user can be a muscle group in the wrist, as one example, but muscle groups further up the user's forearm and elbow, among other areas, can also be targeted from sensing components of a wrist-wearable device that have been repurposed to perform a haptic-rendering function. FIG. 1B also illustrates a chart 114-2 that indicates the biometric signals being recorded by the sensing components 112A-112F over a second time period measured in seconds, i.e., t₁-t₂. A chart 116-2 is also shown in FIG. 1B that indicates measurements of a haptic precursor being applied to a targeted biophysical area of the user over the second time period measured in seconds, i.e., t₁-t₂. As shown with respect to charts 114-2 and 116-2, when the sensing component is repurposed from sensing to directing one or more haptic precursors, the sensing component will temporarily cease sensing a biometric signal. As shown in chart 114-2, at the time that the sensing component 112B directs a haptic precursor (as shown in chart 116-2), the sensing component 112B stops sensing a biometric signal and therefore biometric signal line 113B also stops. The second time period may overlap, in whole or in part, with the first time period or any other time period discussed herein. The second time period may also occur after or before the first time period or any other time period discussed herein. The haptic precursor line 118 shown in chart 116-2 indicates a high frequency (as compared to other example waves shown in subsequent figures) sinusoidal wave with an amplitude that decreases over the second time period as the haptic precursor that is applied to the user 102 via repurposed sensing component 112B. In response to the haptic precursor being applied to the user, a haptic sensation is perceived by a user. In this example, the haptic precursor causes the user's index finger to feel a temporary restriction of movement (e.g., this type of haptic sensation can be provided to the user as the user interacts with objects in an artificial reality to provide a more realistic sensation of holding those objects), as indicated by haptic-sensation waves 120 schematically shown around an index finger 122 of a user 102 to visually represent a location at which the haptic sensation is caused to be felt due to the provision of the haptic precursor at the targeted biophysical area in this example of FIG. 1B. As shown in chart 114-2 and chart 116-2, a haptic precursor can be applied while still recording a biometric signal of the user, which can be referred to as dual-purposing of the sensing components such that they are able to perform sensing and haptic-rendering functions together. This can be achieved by monitoring the haptic precursor and applying a filter to the biometric signal of the user to remove any inaccuracies resulting from the haptic precursor being applied.

FIG. 1C illustrates a third point in time of the user 102 wearing the wearable device 100 where the sensing component 112B is applying a predetermined haptic precursor to a targeted biophysical area of the user (here, targeted muscle group 161), such that the user is caused to perceive a haptic sensation after the haptic precursor is received at the targeted biophysical area of the user. FIG. 1C also illustrates a chart 114-3 that indicates the biometric signals being recorded by the sensing components 112A-112F over a third time period measured in seconds, i.e., t₂-t₃. A chart 116-3 is also shown in FIG. 1C that indicates measurements of a haptic precursor being applied to a targeted biophysical area of the user over the third time period measured in seconds, i.e., t₂-t₃. As shown with respect to charts 114-3 and 116-3, when the sensing component is repurposed from sensing to directing one or more haptic precursors, the sensing component will temporarily cease sensing a biometric signal. As shown in chart 114-3, at the time that the sensing component 112B directs a haptic precursor (as shown in chart 116-3), the sensing component 112B stops sensing a biometric signal and therefore biometric signal line 113B also stops. The third time period may overlap, in whole or in part, with the first or second time periods or any other time period discussed herein. The third time period may also occur after or before the first or second time periods or any other time period discussed herein. The haptic precursor line 117 shown in chart 116-3 indicates a lower frequency sinusoidal wave (e.g., a sinusoidal wave with a frequency that is lower than the frequency shown in chart 116-2 of FIG. 1B) as the haptic precursor that is applied to the user 102. In response to the haptic precursor being applied to the targeted biophysical area of the user, a haptic sensation is perceived by the user. In this example, the user's finger is perceived by the user as feeling a vibration sensation with an associated frequency that, in this example, is visually represented by vibration waves 124 around an index finger 122 of a user 102. As shown in chart 114-3 and chart 116-3, a haptic precursor can be applied while still recording a biometric signal of the user.

FIG. 1D illustrates a fourth point in time of the user 102 wearing the wearable device 100 where the sensing component 112C is applying a predetermined haptic precursor to a different targeted biophysical area of the user (e.g., a muscle group in the wrist) that is different from the biophysical area of the user targeted by sensing component 112B as shown, for example, in FIGS. 1B and 1C such that the user is caused to perceive a haptic sensation in a location that is different than the location discussed in connection with FIGS. 1B and 1C after the haptic precursor is received at the different targeted biophysical area of the user. FIG. 1D also illustrates a chart 114-4 that indicates the biometric signals being recorded by the sensing components 112A-112F over a fourth time period measured in seconds, i.e., t₃-t₄. A chart 116-4 is also shown in FIG. 1D that indicates measurements of a haptic precursor being applied to the different targeted biophysical area of the user over the fourth time period measured in seconds, i.e., t₃-t₄. As shown with respect to charts 114-4 and 116-4, when the sensing component is repurposed from sensing to directing one or more haptic precursors, the sensing component will temporarily cease sensing a biometric signal. As shown in chart 114-4, at the time that the sensing component 112C directs a haptic precursor (as shown in chart 116-4), the sensing component 112C stops sensing a biometric signal and therefore biometric signal line 113C also stops. The fourth time period may overlap, in whole or in part, with the first, second, or third time periods or any other time period discussed herein. The fourth time period may also occur after or before the first, second, or third time periods or any other time period discussed herein. The haptic precursor line 119 shown in chart 116-4 indicates an example frequency sinusoidal wave as haptic precursor that is applied to the user 102. In some embodiments, the haptic precursor line 119 shown in chart 116-4 has the same frequency as haptic precursor line 117 in chart 116-3 of FIG. 1C. In some embodiments, other frequencies can be used to vary the haptic feedbacks; for example, the haptic precursor line 119 and associated frequency, amplitude, and/or phase of the haptic precursor may be higher, lower, or equal to the frequency, amplitude, and/or phase of any other haptic precursor at the same or different targeted biophysical areas of the user. In response to the haptic precursor being applied to the user, a haptic sensation is perceived by a user. In this example, the user's finger may vibrate with an associated frequency as shown by vibration waves 126 around finger 128 of a user 102. The associated frequency of the vibration waves 126 as shown in FIG. 1D may be the same or may be different as the vibrations shown and discussed with respect to FIGS. 1B and 1C, depending at least on the applicable haptic precursor line 118 and the targeted biophysical area of the user. As shown in chart 114-4 and chart 116-4, a haptic precursor can be applied while still recording a biometric signal of the user. This can be achieved by monitoring the haptic precursor and applying a filter to the biometric signal of the user to remove any inaccuracies resulting from the haptic precursor being applied.

FIG. 1E illustrates a fifth point in time of the user 102 wearing the wearable device 100 where two sensing components 112A and 112B are each applying a predetermined haptic precursor to different respective targeted biophysical areas of the user (here, targeted muscle groups 160, 161) such that the user is caused to perceive a haptic sensation at a targeted biophysical area of the user and to concurrently cause movement of a portion of the body (e.g., a finger) of the user 102. FIG. 1E also illustrates a chart 114-5 that indicates the biometric signals being recorded by the sensing components 112A-112F over a fifth time period measured in seconds, i.e., t₄-t₅. A chart 116-5 is also shown in FIG. 1E that indicates measurements of a haptic precursor being applied to a targeted biophysical area of the user that causes the user to perceive a haptic sensation at a targeted biophysical area of the user over the fifth time period measured in seconds, i.e., t₄-t₅. As shown with respect to charts 114-5 and 116-5, when a sensing component is repurposed from sensing to directing one or more haptic precursors, the sensing component will temporarily cease sensing a biometric signal. As shown in chart 114-5, at the time that the sensing components 112A and 112B direct a haptic precursor (as shown in chart 116-5), the sensing components 112A and 112B stop sensing a biometric signal and therefore biometric signal lines 113A and 113B also stop. As shown in chart 116-5, sensing component 112A begins directing a haptic precursor before sensing component 112B. As a result, the sensing component 112A stops sensing a biometric signal before sensing component 112B and the biometric signal line 113A stops before the biometric signal line 113B. The fifth time period may overlap, in whole or in part, with the first, second, third, or fourth time periods or any other time period discussed herein. The fifth time period may also occur after or before the first, second, third, or fourth time periods or any other time period discussed herein. The haptic precursor line 121 shown in chart 116-5 indicates an example frequency sinusoidal wave as haptic precursor that is applied to the user 102 by, in this example, sensing component 112B. In some embodiments, the haptic precursor line 121 shown in chart 116-5 of FIG. 1E has the same frequency as haptic precursor line 117 in chart 116-3 of FIG. 1C. In some embodiments, other frequencies can be used to vary the haptic feedbacks; for example, the haptic precursor line 121 and associated frequency, amplitude, and/or phase of the haptic precursor may be higher, lower, or equal to the frequency, amplitude, and/or phase of any other haptic precursor at the same or different targeted biophysical areas of the user. In response to a haptic precursor being applied to the user, for example by sensing component 112B, a haptic sensation is perceived by a user. By way of the example embodiment shown in FIG. 1E, the haptic precursor applied by sensing component 112B at a targeted biophysical area causes the user's index finger 122 to vibrate. In this example, the user's finger may vibrate with an associated frequency as shown by vibration waves 130 around an index finger 122 of a user 102. The associated frequency of the vibration waves 130 as shown in FIG. 1D may be the same or may be different as the vibrations shown and discussed with respect to any other figure or example embodiment discussed herein, depending at least on the applicable haptic precursor line and targeted biophysical area of the user. As shown in chart 114-5 and chart 116-5, a haptic precursor can be applied while still recording a biometric signal of the user. This can be achieved by monitoring the haptic precursor and applying a filter to the biometric signal of the user to remove any inaccuracies resulting from the haptic precursor being applied.

In response to a haptic precursor being applied to the user at a different targeted biophysical area of the user, for example by sensing component 112A, the user's body may perform an action, such as movement (laterally, vertically, diagonally, in circles, etc.), bending, extension, or otherwise. In some embodiments, the movement can be a restrictive movement by activating a tendon in the user (e.g., to emulate touching an object). As shown in chart 116-5, a second haptic precursor is shown by second haptic precursor line 125, which indicates a constant current being applied to the user 102 by sensing component 112B. As also shown in the example embodiment shown in FIG. 1E, the haptic precursor applied by sensing component 112A at a different targeted biophysical area causes the user's index finger 122 to bend or restrict motion. The haptic precursor applied by sensing component 112A can be applied in such a way as to cause the user's finger to bend (or otherwise moves) more, less, or the same as shown in FIG. 1E. The rate at which the user's finger bends (or otherwise moves) can also be controlled based on the application of the haptic precursor. While the described example shows two discrete haptic precursors to cause the desired haptic feedback, it is possible that the sensing components apply haptic precursors that interfere with each other (e.g., constructively or destructively) to produce more complex haptic feedbacks, including a combination of one or more of muscle activation, tendon activation, skin sensation, and haptic feedbacks.

FIG. 1F illustrates a sixth point in time of the user 102 wearing the wearable device 100 where two sensing components 112E and 112F are each applying a predetermined haptic precursor to different respective targeted biophysical areas of the user (here, targeted muscle groups 163, 164) such that the user is caused to perceive a haptic sensation at a targeted biophysical area of the user. FIG. 1F also illustrates a chart 114-6 that indicates the biometric signals being recorded by the sensing components 112A-112F over a sixth time period measured in seconds, i.e., t₅-t₆. A chart 116-6 is also shown in FIG. 1F that indicates measurements of a haptic precursor being applied to a targeted biophysical area of the user that causes the user to perceive a skin sensation at a targeted biophysical area of the user over the sixth time period measured in seconds, i.e., t₅-t₆. As shown with respect to charts 114-6 and 116-6, when a sensing component is repurposed from sensing to directing one or more haptic precursors, the sensing component will temporarily cease sensing a biometric signal. As shown in chart 114-6, at the time that the sensing components 112E and 112F direct a haptic precursor (as shown in chart 116-6), the sensing components 112E and 112F stop sensing a biometric signal and therefore biometric signal lines 113E and 113F also stop. As also shown in chart 114-6, when the sensing components 112E and 112F stop directing haptic precursors, the sensing components 112E and 112F resume sensing a biometric signal as shown in biometric signal lines 113E and 113F. The sixth time period may overlap, in whole or in part, with the first, second, third, fourth, or fifth time periods or any other time period discussed herein. The sixth time period may also occur after or before the first, second, third, fourth, or fifth time periods or any other time period discussed herein. The haptic precursor line 123 shown in chart 116-6 indicates an example frequency sinusoidal wave as the haptic precursor that is applied to the user 102 at, in this example, sensing component 112E and 112F. In some embodiments, the haptic precursor line 123 and associated frequency sinusoidal wave might be the same, or might be different, from other haptic precursor lines associated with one or more sensing components. In some embodiments, other frequencies can be used to vary the haptic feedbacks; for example, the haptic precursor line 123 and associated frequency, amplitude, and/or phase of the haptic precursor may be higher, lower, or equal to the frequency, amplitude, and/or phase of any other haptic precursor at the same or different targeted biophysical areas of the user. In some embodiments, the sensing components apply haptic precursors that interfere with each other (e.g., constructively or destructively) to produce more complex haptic sensations. In response to the haptic precursor being applied to the user, a haptic sensation is perceived by a user. In this example, the user might experience a skin-feedback sensation as shown by sensation waves 136 around a ring finger 132 or by sensation waves 138 around a pinky finger 134 of a user 102. More specifically, in this non-limiting example, the skin-feedback sensation shown by sensation waves 136 at the ring finger 132 of the user 102 is associated with the haptic precursor applied by sensing component 112E; and the skin-feedback sensation shown by sensation waves 138 at the pinky finger 134 of the user 102 is associated with the haptic precursor applied by sensing component 112F (though in application, the location, haptic precursor characteristics, etc., may be different). As shown in chart 114-6 and chart 116-6, a haptic precursor can be applied while still recording a biometric signal of the user. This can be achieved by monitoring the haptic precursor and applying a filter to the biometric signal of the user to remove any inaccuracies resulting from the haptic precursor being applied.

FIG. 1G illustrates a seventh point in time of the user 102 wearing the wearable device 100 where the sensing component 112F is applying a predetermined haptic precursor to a targeted biophysical area of the user (e.g., a targeted muscle group 164 in the wrist). FIG. 1G also illustrates a chart 114-7 that indicates the biometric signals being recorded by the sensing components 112A-112F over a seventh time period measured in seconds, i.e., t₆-t₇. A chart 116-7 is also shown in FIG. 1G that indicates measurements of a haptic precursor being applied to the different targeted biophysical area of the user over the seventh time period measured in seconds, i.e., t₆-t₇. As shown with respect to charts 114-7 and 116-7, when the sensing component is repurposed from sensing to directing one or more haptic precursors, the sensing component will temporarily cease sensing a biometric signal. As shown in chart 114-7, at the time that the sensing component 112F directs a haptic precursor (as shown in chart 116-7), the sensing component 112F stops sensing a biometric signal and therefore biometric signal line 113D also stops. The seventh time period may overlap, in whole or in part, with the fourth, fifth, or sixth time periods or any other time period discussed herein. The seventh time period may also occur after or before the fourth, fifth, or sixth time periods or any other time period discussed herein. The haptic precursor line 140 shown in chart 116-7 indicates an example frequency sinusoidal wave as haptic precursor that is applied to the user 102. In some embodiments, the haptic precursor line 140 shown in chart 116-7 has the same frequency as haptic precursor line 117 in chart 116-3 of FIG. 1C. In some embodiments, other frequencies can be used to vary the haptic feedbacks; for example, the haptic precursor line 140 and associated frequency, amplitude, and/or phase of the haptic precursor may be higher, lower, or equal to the frequency, amplitude, and/or phase of any other haptic precursor at the same or different targeted biophysical areas of the user. In response to the haptic precursor being applied to the user, a haptic sensation is perceived by a user. In this example, the user's elbow can vibrate with an associated frequency as shown by vibration waves 142 around elbow 144 of a user 102. The associated frequency of the vibration waves 142 as shown in FIG. 1G may be the same or may be different as the vibrations shown and discussed with respect to FIGS. 1B and 1C, depending at least on the applicable haptic precursor line 118 and the targeted biophysical area of the user. As shown in chart 114-7 and chart 116-7, a haptic precursor can be applied while still recording a biometric signal of the user. This can be achieved by monitoring the haptic precursor and applying a filter to the biometric signal of the user to remove any inaccuracies resulting from the haptic precursor being applied.

In the examples discussed above, in some embodiments, a portion of a conductive surface on each of the sensing components 112A-112F from which the haptic precursor is sent can be electrically isolated from a remaining portion of the conductive surface to avoid interrupting signal detection when the haptic precursor is being delivered to a targeted biophysical area. For example, each of the sensing components 112A-112F can be a dual function (e.g., a dual channel) sensing component where one function can be temporarily configured to provide a haptic precursor while the other function remains configured to be a sensing component. In some embodiments, a dual function sensing component can be in a single structure (e.g., electromyography pill or button shape) that has two sensing components that are electrically isolated from each other while both being in the same housing.

The location, frequency, intensity, and association of the haptic precursor and the haptic sensation felt by the user may be user specific. In addition to the sensations discussed with respect to the example embodiments referenced herein, the haptic sensation(s) felt by the user include, but are not limited to, a muscular movement, a nerve stimulation, a tendon activation, and a skin-feedback sensation.

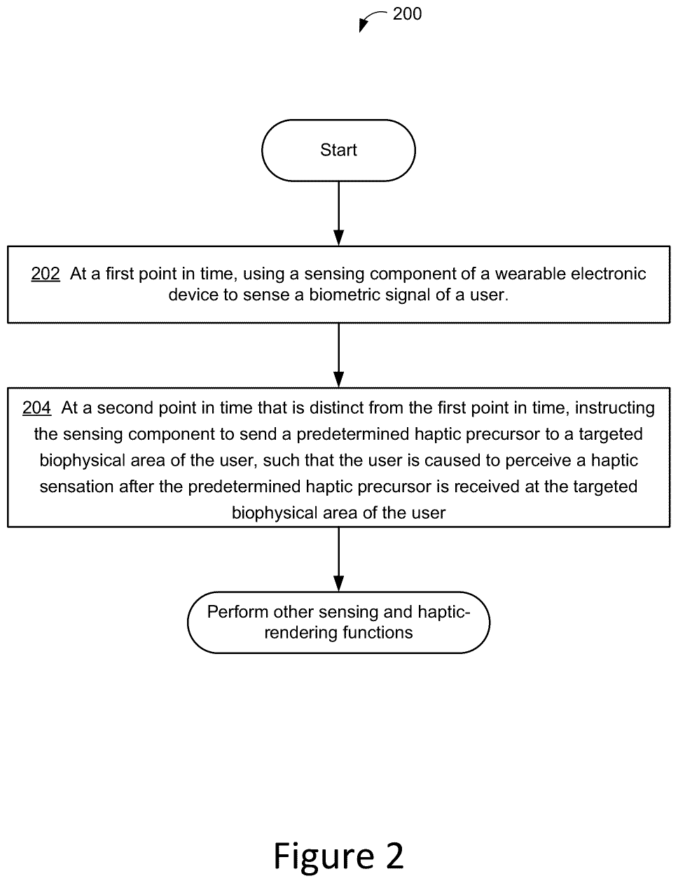

FIG. 2 shows a flow chart of a method 200 of repurposing a sensing component to also perform a haptic-rendering function, in accordance with some embodiments. At 202, the example method includes, at a first point in time, using a sensing component of a wearable electronic device to sense a biometric signal of a user. At 204, the example method further includes at a second point in time that is distinct from the first point in time, instructing the sensing component to send a predetermined haptic precursor to a targeted biophysical area of the user, such that the user is caused to perceive a haptic sensation after the predetermined haptic precursor is received at the targeted biophysical area of the user. The example method proceeds to perform other sensing and haptic rendering functions.
(A1) One example of a method of repurposing a sensing component to perform a haptic-rendering function is described herein. This example method includes, at a first point in time, using a sensing component of a wearable electronic device to sense a biometric signal of a user. The example method further includes, at a second point in time that is distinct from the first point in time, instructing the sensing component to send a predetermined haptic precursor to a targeted biophysical area of the user, such that the user is caused to perceive a haptic sensation after the predetermined haptic precursor is received at the targeted biophysical area of the user. For example, FIGS. 1A-1G show, at a first point in time, sensing a biometric signal of a user (for example, heart rate as shown in charts 114-1 to 114-6) and, at a second point in time, sending a predetermined haptic precursor (for example, as shown in cross-sectional views 106 and charts 116-2 to 116-6).

In some embodiments, the sensing component is configured to sense one or more neuromuscular signals. In some other embodiments, the sensing component is an electrode that may be configured to sense one or more neuromuscular signals. In some embodiments, the haptic precursor is an electrical current sent to a biophysical area of the user. In some embodiments, the electrode is in a differential pair of electrodes that forms a sensing channel for neuromuscular signals.

In some embodiments, the targeted biophysical area may vary from user to user. For example, a particular haptic precursor may cause a different haptic sensation to be perceived if applied to a first user as opposed to if that same precursor was applied at the same targeted biophysical area on a second user. The difference in perceived haptic sensations may be based on a variety of factors specific to the user, including body type, size of the user's wrist, hand, arm, etc., hair, sweat, skin moisture levels, muscle tone, etc. The difference may also be non-user-specific, such as whether the wearable device is donned differently by one user than the other (or from one use to another), or based on the conditions (e.g., heat, humidity, etc.) in which a user is using the apparatus. The wrist-wearable device may be configured to learn user-specific or non-user-specific information regarding a haptic precursor and a corresponding perceived haptic sensation and efficiently adjust later stimulations.
(A2) In some embodiments of A1, the sensing component is an electrode configured to sense one or more neuromuscular signals, and the haptic precursor is an electrical current sent to the biophysical area of the user. For example, at least FIG. 1A shows sensing components 112A-112F, which are examples of electrodes configured to sense one or more neuromuscular signals.
(A3) In some embodiments of any of A1-A2, the example method further includes, at a third point in time that is distinct from the first point in time and the second point in time, instructing the sensing component to send another predetermined haptic precursor to the targeted biophysical area of the user. The other predetermined haptic precursor is different from the haptic precursor, such that the user is caused to perceive another haptic sensation after the haptic precursor is received at the targeted biophysical area of the user. For example, at least FIGS. 1C-1F show sending another predetermined haptic precursor to a targeted biophysical area of the user.

In some embodiments, the second (or other/different) haptic precursor varies from the other haptic precursor at least because it is a haptic precursor that is applied at a different time than the first haptic precursor. In some embodiments, the second haptic precursor may also vary from the first haptic precursor by being applied at a different targeted biophysical area of the user. In some embodiments, the second haptic precursor may also vary from the first haptic precursor by one or more of amplitude, phase, or frequency. In some embodiments, still further haptic precursors—e.g., third, fourth, fifth, etc.—may be applied. In some embodiments, these further haptic precursors may differ from other haptic precursors in one or more of the ways discussed herein, or may differ in other ways as well.

Sending multiple haptic precursors may occur in a variety of ways. In some embodiments, the sensing component and another sensing component is instructed to send another predetermined haptic precursor to the targeted biophysical area of the user, such that the user is caused to perceive the haptic sensation after the haptic precursor is received at the targeted biophysical area of the user. In some embodiments, the same sensing component is instructed to send multiple haptic precursors. In some embodiments, a first sensing component sends a first haptic precursor, and then one or more of a second, third, fourth, etc., sensing component sends respective haptic precursors (at the same or different times) to cause a haptic sensation to be perceived by the user.
(A4) In some embodiments of any of A1-A3, the haptic sensation is one or more of a muscular movement, a nerve stimulation, a tendon activation, and a skin-feedback sensation. For example, at least FIGS. 1A-1G show example haptic sensations (e.g., vibration waves 120, 124, 126, 130 and sensation waves 136, 138).

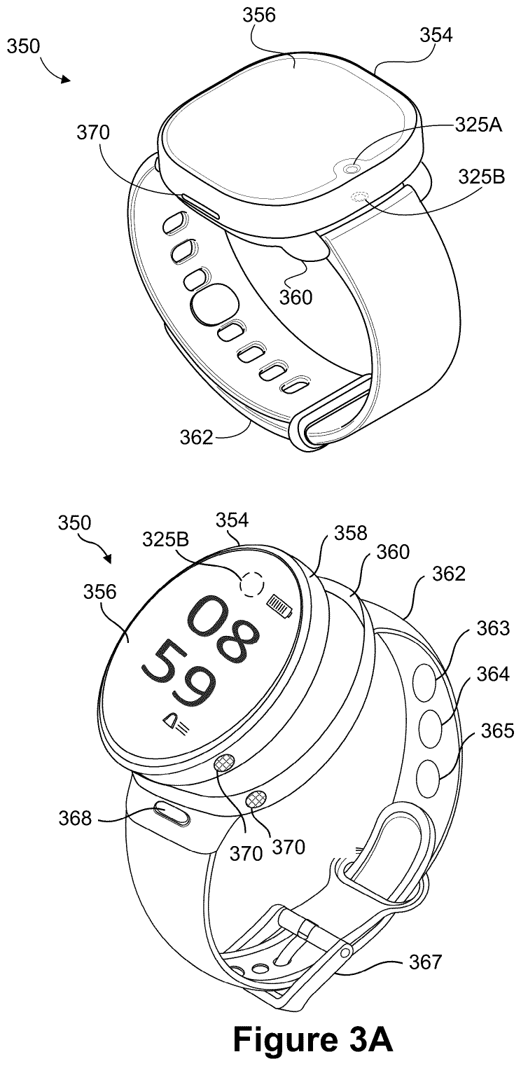

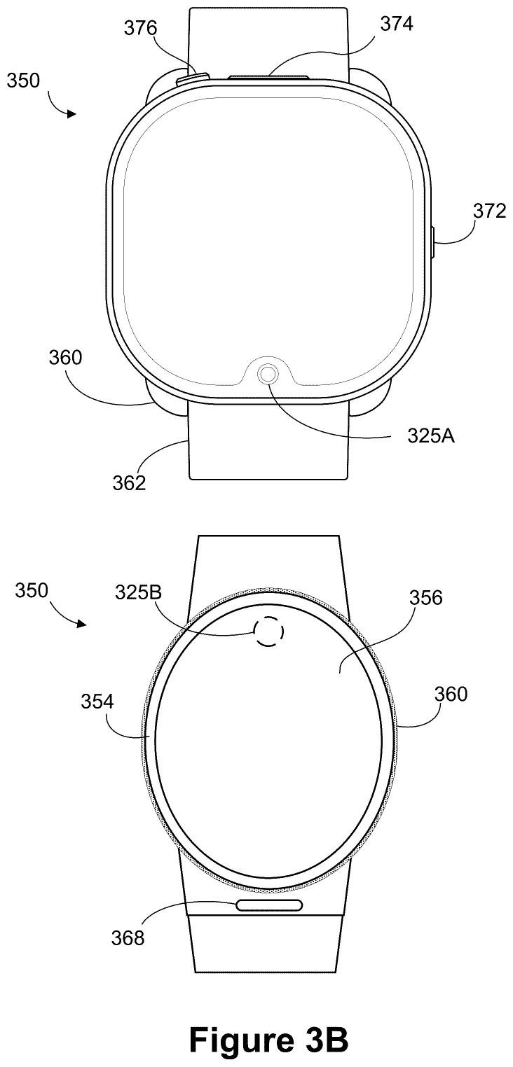

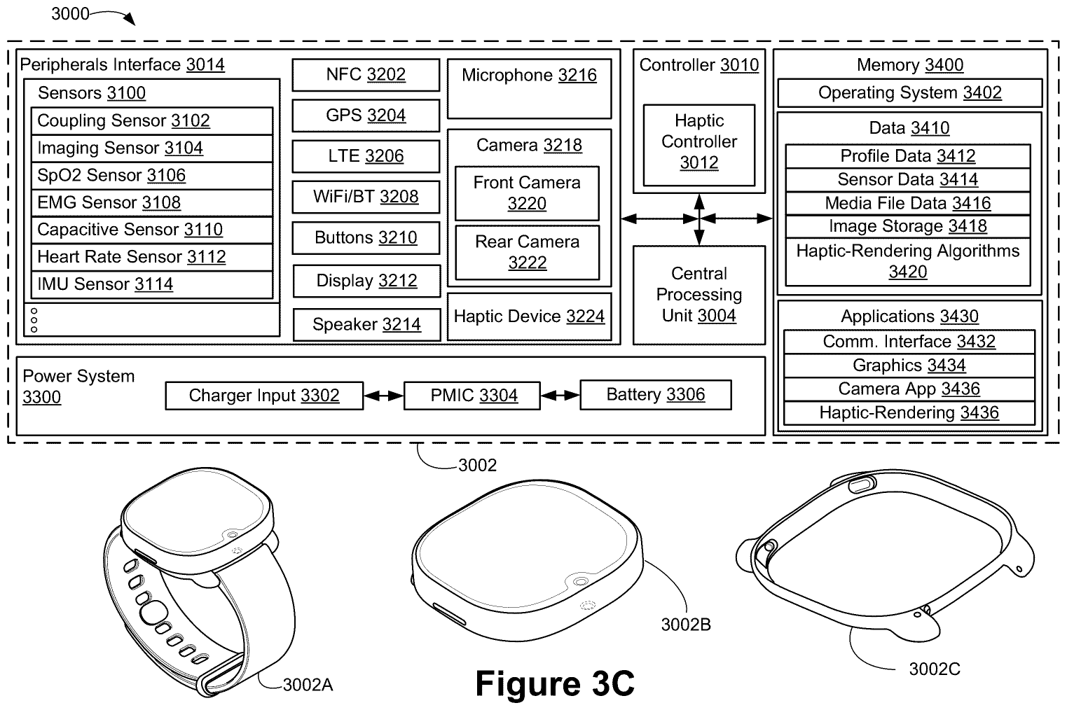

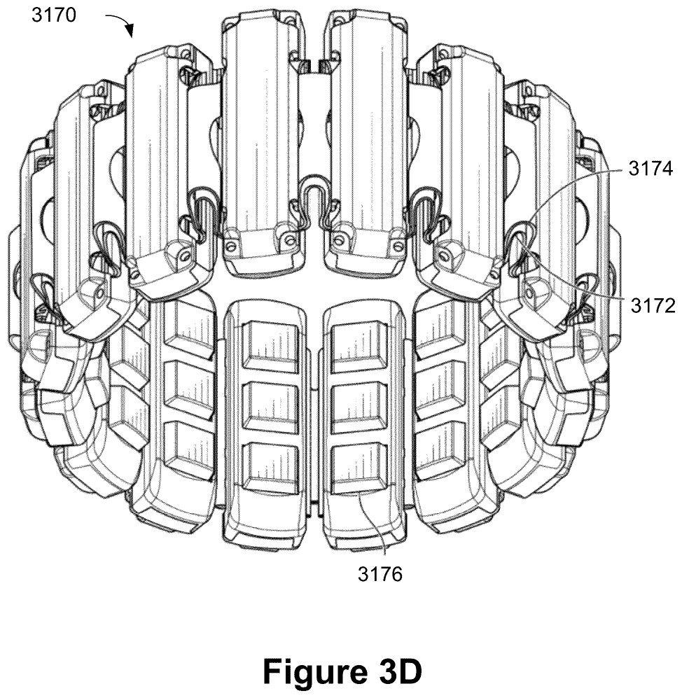

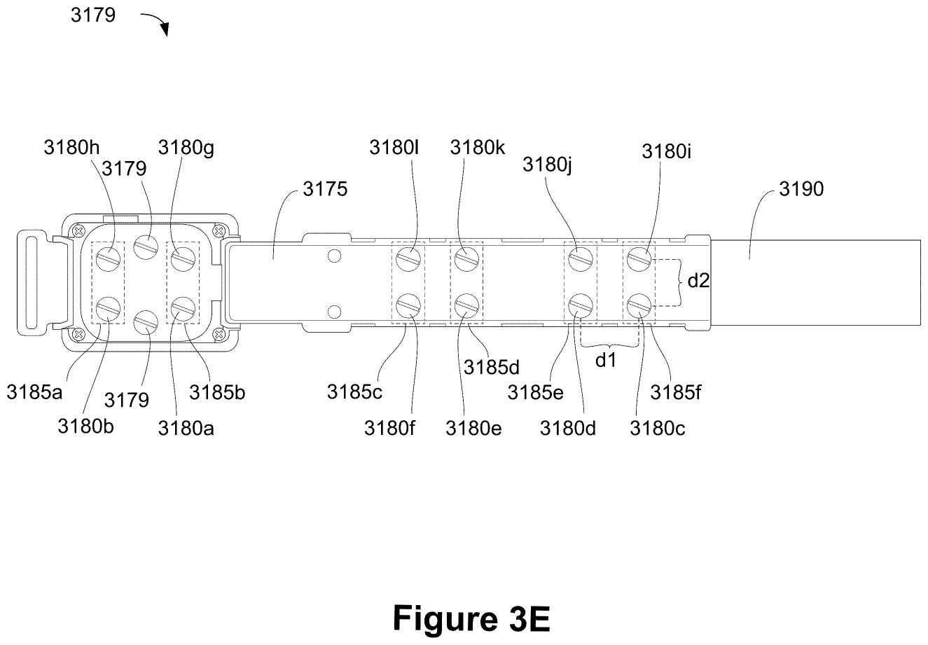

The haptic sensation can include a variety of sensations. In some embodiments, the haptic sensation is one or more of a muscular movement, a nerve stimulation, a tendon activation, and a skin-feedback sensation.
(A5) In some embodiments of any of A1-A4, the sensing component is an electrode in a differential pair of electrodes that forms a sensing channel for neuromuscular signals. For example, at least FIG. 3E shows electrodes 3180 that can be an electrode in a differential pair of electrodes.
(A6) In some embodiments of any of A1-A5, the example method further includes, at a third point in time, instructing the sensing component to send a predetermined haptic precursor to another targeted biophysical area of the user different than the targeted biophysical area, such that the user is caused to perceive a different haptic sensation after the haptic precursor is received at the other targeted biophysical area of the user. For example, at least FIGS. 1D-1F show sending a predetermined haptic precursor to another targeted biophysical area that is different from the area targeted in, for example, FIGS. 1B-1C.
(A7) In some embodiments of any of A1-A6, the example method further includes, at a third point in time, using another sensing component to sense another biometric signal of a user. And it further includes, at a fourth point in time, instructing the other sensing component to send another predetermined haptic precursor to another targeted biophysical area of the user, such that the user is caused to perceive another haptic sensation after the other haptic precursor is received at the targeted biophysical area of the user. For example, at least FIGS. 1D-1F show sending a predetermined haptic precursor to another targeted biophysical area that is different from the area targeted in, for example, FIGS. 1B-1C.
(A8) In some embodiments of any of A1-A7, the example method further includes, at a second point in time that is distinct from the first point in time, instructing both the sensing component and another sensing component to send another predetermined haptic precursor to the targeted biophysical area of the user, such that the user is caused to perceive the haptic sensation after the haptic precursor is received at the targeted biophysical area of the user. For example, FIG. 1E shows two sensing components, 112B and 112A, sending predetermined haptic precursors at a point in time that may be different from the point in time during which another haptic precursor was sent (for example as shown in FIG. 1B from sensing component 112B).
(A9) In some embodiments of any of A1-A8, the example method further includes, at a second point in time that is distinct from the first point in time, instructing the sensing component to send the predetermined haptic precursor to the targeted biophysical area of the user, such that the user is caused to perceive the haptic sensation after the haptic precursor is received at the targeted biophysical area of the user. In some embodiments, the example method further includes, at a second point in time that is distinct from the first point in time, instructing another sensing component to send another haptic precursor to another targeted biophysical area of the user, such that the user is caused to perceive another haptic sensation after the other haptic precursor is received at the other targeted biophysical area of the user. For example, FIGS. 1A-1G show example embodiments where different sensing components (e.g., 112A-112F) can sense a biometric signal and send one or more haptic precursors to respective targeted biophysical areas of the user 102 at different points in time (e.g., t₀ to t₆).
(A10) In some embodiments of any of A1-A9, the user is caused to perceive the haptic sensation without the user wearing a glove. For example, FIGS. 1A-1G show example embodiments where the user is not wearing a glove.

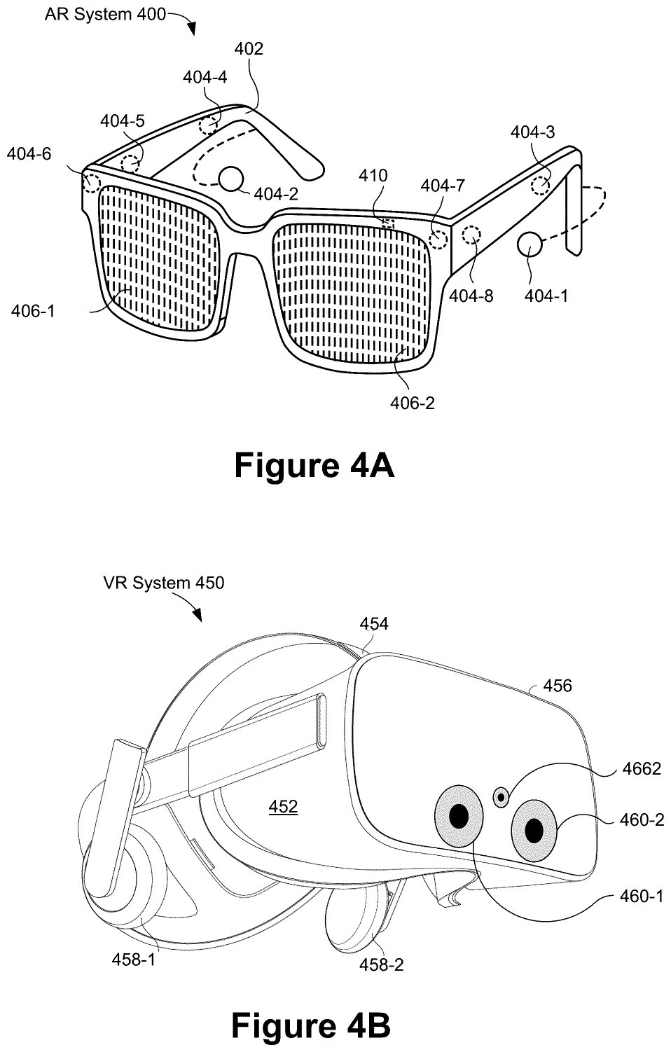

In some embodiments, the user is caused to perceive the haptic sensation through an arm-worn (or arm-wearable) device. In some embodiments, an arm-worn (or arm-wearable) device can include, for example, a wrist-worn wearable device, an upper-forearm-worn band wearable device, an arm-worn band wearable device, a sleeve wearable device, etc. In some embodiments, the arm-worn wearable device includes one or more of these components—for example a wrist-worn device and another ring placed around another part of the user's body (e.g., forearm, upper arm, leg, ankle, or otherwise). In some embodiments, the user is caused to perceive the haptic sensation through the user wearing a glove. In some embodiments, the user is caused to perceive the haptic sensation without the user wearing a glove. In some embodiments, one or more of the sensing components are part of the wearable device(s).
(A11) In some embodiments of any of A1-A10, the wearable electronic device is a wrist-wearable device that is configured to be in communication with a head-wearable device, and the haptic sensation is configured to be provided when a hand of the user is determined to be within a predetermined distance of an object presented to the user via the head-wearable device. For example, FIGS. 1A-1G show example embodiments where the user is wearing a head-wearable device, for example the AR system 400 of FIG. 4A.
(A12) In some embodiments of any of A1-A11, the example method further includes, at a third point in time, instructing both the sensing component and another sensing component to send another predetermined haptic precursor to the targeted biophysical area of the user, such that the user is caused to perceive another haptic sensation, distinct from the haptic sensation, after the other haptic precursor is received at the targeted biophysical area of the user. For example, FIGS. 1A-1G show example embodiments where different sensing components (e.g., 112A-112F) can sense a biometric signal and send one or more haptic precursors to respective targeted biophysical areas of the user 102 at different points in time (e.g., t₀ to t₆).
(A13) In some embodiments of any of A1-A12, the example method further includes, at a fourth point in time, instructing the sensing component to send yet another predetermined haptic precursor to the targeted biophysical area of the user, such that the user is caused to perceive a different haptic sensation after the other predetermined haptic precursor, distinct from the haptic sensation and the other haptic sensation, is received at the targeted biophysical area of the user; and the other sensing component is configured to sense another signal of the user. For example, FIGS. 1A-1G show example embodiments where different sensing components (e.g., 112A-112F) can sense a biometric signal and send one or more haptic precursors to respective targeted biophysical areas of the user 102 at different points in time (e.g., t₀ to t₆).
(A14) In some embodiments of any of A1-A13, the sensing component is part of an arm-worn wearable device. For example, FIGS. 1A-1G show a wrist-wearable device 100. Example wrist-wearable devices are shown in, for example, FIGS. 3A-3E.
(A15) In some embodiments of any of A1-A9 and A11-A14, the sensing component is part of a glove-worn wearable device.
(A16) In some embodiments of any of A1-15, the sensing component can send the haptic precursor to the targeted biophysical area of the user in conjunction with activation of a different type of haptic feedback generator to collectively provide another haptic sensation different from the haptic sensation. For example, FIGS. 1A-1G show example embodiments where different sensing components (e.g., 112A-112F) can sense a biometric signal and send one or more haptic precursors to respective targeted biophysical areas of the user 102 at different points in time (e.g., t₀ to t₆).
(A17) In some embodiments of any of A1-A18, the haptic precursor is configured to mimic natural nervous system firing patterns. For example, the example haptic precursors shown in graphs 116-2 to 116-6 of FIGS. 1B-1F may mimic natural nervous system firing patterns.

In some embodiments, the haptic precursor is configured to mimic natural nervous system firing patterns. In some embodiments, the haptic precursor is configured to be applied to one or both of a medial or ulnar nerve of a user. In some embodiments, the biometric signal of the user is not corrupted when the haptic precursor is applied.
(A18) In some embodiments of any of A1-A17, the haptic precursor is configured to be applied to one or both of a medial or ulnar nerve of a user. For example, FIGS. 1B-1F show a haptic precursor being applied to a portion of the wrist of user 102 that may be one or both of a medial or ulnar nerve of the user.
(A19) In some embodiments of any of A1-A18, the biometric signal of the user is not corrupted when the haptic precursor is applied. For example, FIGS. 1B-1F show that a biometric signal (e.g. heart rate as shown in graph 114-6) is not corrupted when a haptic precursor is applied.
(B1) In another aspect example described herein, a wearable device is provided. The example wearable device is capable of being configured to perform any of the methods of A1-A19.
(C1) In another aspect example described herein, a system is provided. The example system includes a wearable device and a head-wearable display device. The system is configured to present user interfaces via the head-wearable device. The wearable device is capable of being configured to perform any of the methods of A1-A19.
(D1) In another aspect example described herein, a non-transitory, computer-readable storage medium including instructions is provided. The non-transitory, computer-readable storage medium includes instructions that, when executed by a wrist-wearable device, cause the wrist-wearable device to perform or cause performance of any of the methods of A1-A19.
(E1) In one further aspect, means for performing the method of any of A1-A19 is provided.

The devices described above are further detailed below, including wrist-wearable devices, headset devices, systems, and haptic feedback devices. Specific operations described above may occur as a result of specific hardware; such hardware is described in further detail below. The devices described below are not limiting and features on these devices can be removed or additional features can be added to these devices.

### Example Wrist-Wearable Devices

FIGS. 3A and 3B illustrate an example wrist-wearable device 350, in accordance with some embodiments. The wrist-wearable device 350 is an instance of the wearable device described herein, such that the wearable device should be understood to have the features of the wrist-wearable device 350 and vice versa. FIG. 3A illustrates a perspective view of the wrist-wearable device 350 that includes a watch body 354 coupled with a watch band 362. The watch body 354 and the watch band 362 can have a substantially rectangular or circular shape and can be configured to allow a user to wear the wrist-wearable device 350 on a body part (e.g., a wrist). The wrist-wearable device 350 can include a retaining mechanism 367 (e.g., a buckle, a hook and loop fastener, etc.) for securing the watch band 362 to the user's wrist. The wrist-wearable device 350 can also include a coupling mechanism 360 (e.g., a cradle) for detachably coupling the capsule or watch body 354 (via a coupling surface of the watch body 354) to the watch band 362.

The wrist-wearable device 350 can perform various functions associated with navigating through user interfaces and selectively opening applications, as described herein. As will be described in more detail below, operations executed by the wrist-wearable device 350 can include, without limitation, display of visual content to the user (e.g., visual content displayed on display 356); sensing user input (e.g., sensing a touch on peripheral button 368, sensing biometric data on sensor 364, sensing neuromuscular signals on neuromuscular sensor 365, etc.); messaging (e.g., text, speech, video, etc.); image capture; wireless communications (e.g., cellular, near field, Wi-Fi, personal area network, etc.); location determination; financial transactions; providing haptic feedback; alarms; notifications; biometric authentication; health monitoring; sleep monitoring; etc. These functions can be executed independently in the watch body 354, independently in the watch band 362, and/or in communication between the watch body 354 and the watch band 362. In some embodiments, functions can be executed on the wrist-wearable device 350 in conjunction with an artificial-reality environment that includes, but is not limited to, virtual-reality (VR) environments (including non-immersive, semi-immersive, and fully immersive VR environments); augmented-reality environments (including marker-based augmented-reality environments, markerless augmented-reality environments, location-based augmented-reality environments, and projection-based augmented-reality environments); hybrid reality; and other types of mixed-reality environments. As the skilled artisan will appreciate upon reading the descriptions provided herein, the novel wearable devices described herein can be used with any of these types of artificial-reality environments.

The watch band 362 can be configured to be worn by a user such that an inner surface of the watch band 362 is in contact with the user's skin. When worn by a user, sensor 364 is in contact with the user's skin. The sensor 364 can be a biosensor that senses a user's heart rate, saturated oxygen level, temperature, sweat level, muscle intentions, or a combination thereof. The watch band 362 can include multiple sensors 364 that can be distributed on an inside and/or an outside surface of the watch band 362. Additionally, or alternatively, the watch body 354 can include sensors that are the same or different than those of the watch band 362 (or the watch band 362 can include no sensors at all in some embodiments). For example, multiple sensors can be distributed on an inside and/or an outside surface of the watch body 354. As described below with reference to FIGS. 3B and/or 3C, the watch body 354 can include, without limitation, a front-facing image sensor 325A and/or a rear-facing image sensor 325B, a biometric sensor, an inertial measurement unit (IMU), a heart rate sensor, a saturated oxygen sensor, a neuromuscular sensor(s), an altimeter sensor, a temperature sensor, a bioimpedance sensor, a pedometer sensor, an optical sensor (e.g., imaging sensor 3104), a touch sensor, a sweat sensor, etc. The sensor 364 can also include a sensor that provides data about a user's environment including a user's motion (e.g., an IMU), altitude, location, orientation, gait, or a combination thereof. The sensor 364 can also include a light sensor (e.g., an infrared light sensor, a visible light sensor) that is configured to track a position and/or motion of the watch body 354 and/or the watch band 362. The watch band 362 can transmit the data acquired by sensor 364 to the watch body 354 using a wired communication method (e.g., a Universal Asynchronous Receiver/Transmitter (UART), a USB transceiver, etc.) and/or a wireless communication method (e.g., near field communication, Bluetooth, etc.). The watch band 362 can be configured to operate (e.g., to collect data using sensor 364) independent of whether the watch body 354 is coupled to or decoupled from watch band 362.

In some examples, the watch band 362 can include a neuromuscular sensor 365 (e.g., an EMG sensor, a mechanomyogram (MMG) sensor, a sonomyography (SMG) sensor, etc.). Neuromuscular sensor 365 can sense a user's intention to perform certain motor actions. The sensed muscle intention can be used to control certain user interfaces displayed on the display 356 of the wrist-wearable device 350 and/or can be transmitted to a device responsible for rendering an artificial-reality environment (e.g., a head-mounted display) to perform an action in an associated artificial-reality environment, such as to control the motion of a virtual device displayed to the user.

Signals from neuromuscular sensor 365 can be used to provide a user with an enhanced interaction with a physical object and/or a virtual object in an artificial-reality application generated by an artificial-reality system (e.g., user interface objects presented on the display 356, or another computing device (e.g., a smartphone)). Signals from neuromuscular sensor 365 can be obtained (e.g., sensed and recorded) by one or more neuromuscular sensors 365 of the watch band 362. Although FIG. 3A shows one neuromuscular sensor 365, the watch band 362 can include a plurality of neuromuscular sensors 365 arranged circumferentially on an inside surface of the watch band 362 such that the plurality of neuromuscular sensors 365 contact the skin of the user. The watch band 362 can include a plurality of neuromuscular sensors 365 arranged circumferentially on an inside surface of the watch band 362. Neuromuscular sensor 365 can sense and record neuromuscular signals from the user as the user performs muscular activations (e.g., movements, gestures, etc.). The muscular activations performed by the user can include static gestures, such as placing the user's hand palm down on a table; dynamic gestures, such as grasping a physical or virtual object; and covert gestures that are imperceptible to another person, such as slightly tensing a joint by co-contracting opposing muscles or using sub-muscular activations. The muscular activations performed by the user can include symbolic gestures (e.g., gestures mapped to other gestures, interactions, or commands, for example, based on a gesture vocabulary that specifies the mapping of gestures to commands).

The watch band 362 and/or watch body 354 can include a haptic device 363 (e.g., a vibratory haptic actuator) that is configured to provide haptic feedback (e.g., a cutaneous and/or kinesthetic sensation, etc.) to the user's skin. The sensors 364 and 365, and/or the haptic device 363, can be configured to operate in conjunction with multiple applications including, without limitation, health monitoring, social media, game playing, and artificial reality (e.g., the applications associated with artificial reality).

The wrist-wearable device 350 can include a coupling mechanism (also referred to as a cradle) for detachably coupling the watch body 354 to the watch band 362. A user can detach the watch body 354 from the watch band 362 in order to reduce the encumbrance of the wrist-wearable device 350 to the user. The wrist-wearable device 350 can include a coupling surface on the watch body 354 and/or coupling mechanism(s) 360 (e.g., a cradle, a tracker band, a support base, a clasp). A user can perform any type of motion to couple the watch body 354 to the watch band 362 and to decouple the watch body 354 from the watch band 362. For example, a user can twist, slide, turn, push, pull, or rotate the watch body 354 relative to the watch band 362, or a combination thereof, to attach the watch body 354 to the watch band 362 and to detach the watch body 354 from the watch band 362.

As shown in the example of FIG. 3A, the watch band coupling mechanism 360 can include a type of frame or shell that allows the watch body 354 coupling surface to be retained within the watch band coupling mechanism 360. The watch body 354 can be detachably coupled to the watch band 362 through a friction fit, magnetic coupling, a rotation-based connector, a shear-pin coupler, a retention spring, one or more magnets, a clip, a pin shaft, a hook and loop fastener, or a combination thereof. In some examples, the watch body 354 can be decoupled from the watch band 362 by actuation of the release mechanism 370. The release mechanism 370 can include, without limitation, a button, a knob, a plunger, a handle, a lever, a fastener, a clasp, a dial, a latch, or a combination thereof.

As shown in FIGS. 3A-3B, the coupling mechanism 360 can be configured to receive a coupling surface proximate to the bottom side of the watch body 354 (e.g., a side opposite to a front side of the watch body 354 where the display 356 is located), such that a user can push the watch body 354 downward into the coupling mechanism 360 to attach the watch body 354 to the coupling mechanism 360. In some embodiments, the coupling mechanism 360 can be configured to receive a top side of the watch body 354 (e.g., a side proximate to the front side of the watch body 354 where the display 356 is located) that is pushed upward into the cradle, as opposed to being pushed downward into the coupling mechanism 360. In some embodiments, the coupling mechanism 360 is an integrated component of the watch band 362 such that the watch band 362 and the coupling mechanism 360 are a single unitary structure.

The wrist-wearable device 350 can include a single release mechanism 370 or multiple release mechanisms 370 (e.g., two release mechanisms 370 positioned on opposing sides of the wrist-wearable device 350 such as spring-loaded buttons). As shown in FIG. 3A, the release mechanism 370 can be positioned on the watch body 354 and/or the watch band coupling mechanism 360. Although FIG. 3A shows release mechanism 370 positioned at a corner of watch body 354 and at a corner of watch band coupling mechanism 360, the release mechanism 370 can be positioned anywhere on watch body 354 and/or watch band coupling mechanism 360 that is convenient for a user of wrist-wearable device 350 to actuate. A user of the wrist-wearable device 350 can actuate the release mechanism 370 by pushing, turning, lifting, depressing, shifting, or performing other actions on the release mechanism 370. Actuation of the release mechanism 370 can release (e.g., decouple) the watch body 354 from the watch band coupling mechanism 360 and the watch band 362 allowing the user to use the watch body 354 independently from watch band 362. For example, decoupling the watch body 354 from the watch band 362 can allow the user to capture images using rear-facing image sensor 325B.

FIG. 3B includes top views of examples of the wrist-wearable device 350. The examples of the wrist-wearable device 350 shown in FIGS. 3A-3B can include a coupling mechanism 360 (as shown in FIG. 3B, the shape of the coupling mechanism can correspond to the shape of the watch body 354 of the wrist-wearable device 350). The watch body 354 can be detachably coupled to the coupling mechanism 360 through a friction fit, magnetic coupling, a rotation-based connector, a shear-pin coupler, a retention spring, one or more magnets, a clip, a pin shaft, a hook and loop fastener, or any combination thereof.

In some examples, the watch body 354 can be decoupled from the coupling mechanism 360 by actuation of a release mechanism 370. The release mechanism 370 can include, without limitation, a button, a knob, a plunger, a handle, a lever, a fastener, a clasp, a dial, a latch, or a combination thereof. In some examples, the wristband system functions can be executed independently in the watch body 354, independently in the coupling mechanism 360, and/or in communication between the watch body 354 and the coupling mechanism 360. The coupling mechanism 360 can be configured to operate independently (e.g., execute functions independently) from watch body 354. Additionally, or alternatively, the watch body 354 can be configured to operate independently (e.g., execute functions independently) from the coupling mechanism 360. As described below with reference to the block diagram of FIG. 3A, the coupling mechanism 360 and/or the watch body 354 can each include the independent resources required to independently execute functions. For example, the coupling mechanism 360 and/or the watch body 354 can each include a power source (e.g., a battery), a memory, data storage, a processor (e.g., a central processing unit (CPU)), communications, a light source, and/or input/output devices.

The wrist-wearable device 350 can have various peripheral buttons 372, 374, and 376, for performing various operations at the wrist-wearable device 350. Also, various sensors, including one or both of the sensors 364 and 365, can be located on the bottom of the watch body 354, and can optionally be used even when the watch body 354 is detached from the watch band 362.

FIG. 3C is a block diagram of a computing system 3000, according to at least one embodiment of the present disclosure. The computing system 3000 includes an electronic device 3002, which can be, for example, a wrist-wearable device. The wrist-wearable device 350 described in detail above with respect to FIGS. 3A-3B is an example of the electronic device 3002, so the electronic device 3002 will be understood to include the components shown and described below for the computing system 3000. In some embodiments, all, or a substantial portion, of the components of the computing system 3000 are included in a single integrated circuit. In some embodiments, the computing system 3000 can have a split architecture (e.g., a split mechanical architecture, a split electrical architecture) between a watch body (e.g., a watch body 354 in FIGS. 3A-3B) and a watch band (e.g., a watch band 362 in FIGS. 3A-3B). The electronic device 3002 can include a processor (e.g., a central processing unit 3004), a controller 3010, a peripherals interface 3014 that includes one or more sensors 3100 and various peripheral devices, a power source (e.g., a power system 3300), and memory (e.g., a memory 3400) that includes an operating system (e.g., an operating system 3402), data (e.g., data 3410), and one or more applications (e.g., applications 3430).

In some embodiments, the computing system 3000 includes the power system 3300, which includes a charger input 3302, a power-management integrated circuit (PMIC) 3304, and a battery 3306.

In some embodiments, a watch body and a watch band can each be electronic devices 3002 that each have respective batteries (e.g., battery 3306) and can share power with each other. The watch body and the watch band can receive a charge using a variety of techniques. In some embodiments, the watch body and the watch band can use a wired charging assembly (e.g., power cords) to receive the charge. Alternatively, or in addition, the watch body and/or the watch band can be configured for wireless charging. For example, a portable charging device can be designed to mate with a portion of the watch body and/or the watch band and wirelessly deliver usable power to a battery of the watch body and/or the watch band.

The watch body and the watch band can have independent power systems 3300 to enable each to operate independently. The watch body and the watch band can also share power (e.g., one can charge the other) via respective PMICs 3304 that can share power over power and ground conductors and/or over wireless charging antennas.

In some embodiments, the peripherals interface 3014 can include one or more sensors 3100. The sensors 3100 can include a coupling sensor 3102 for detecting when the electronic device 3002 is coupled with another electronic device 3002 (e.g., a watch body can detect when it is coupled to a watch band, and vice versa). The sensors 3100 can include imaging sensors 3104 for collecting imaging data, which can optionally be the same device as one or more of the cameras 3218. In some embodiments, the imaging sensors 3104 can be separate from the cameras 3218. In some embodiments, the sensors include an SpO2 sensor 3106. In some embodiments, the sensors 3100 include an EMG sensor 3108 for detecting, for example, muscular movements by a user of the electronic device 3002. In some embodiments, the sensors 3100 include a capacitive sensor 3110 for detecting changes in potential of a portion of a user's body. In some embodiments, the sensors 3100 include a heart rate sensor 3112. In some embodiments, the sensors 3100 include an IMU sensor 3114 for detecting, for example, changes in acceleration of the user's hand.

In some embodiments, the peripherals interface 3014 includes a near-field communication (NFC) component 3202, a global-position system (GPS) component 3204, a long-term evolution (LTE) component 3206, and/or a Wi-Fi or Bluetooth communication component 3208.

In some embodiments, the peripherals interface includes one or more buttons (e.g., the peripheral buttons 357, 358, and 359 in FIG. 3B) that, when selected by a user, cause operation to be performed at the electronic device 3002.

The electronic device 3002 can include at least one display 3212, for displaying visual affordances to the user, including user-interface elements and/or three-dimensional virtual objects. The display can also include a touch screen for inputting user inputs, such as touch gestures, swipe gestures, and the like.

The electronic device 3002 can include at least one speaker 3214 and at least one microphone 3216 for providing audio signals to the user and receiving audio input from the user. The user can provide user inputs through the microphone 3216 and can also receive audio output from the speaker 3214 as part of a haptic event provided by the haptic controller 3012.

The electronic device 3002 can include at least one camera 3218, including a front camera 3220 and a rear camera 3222. In some embodiments, the electronic device 3002 can be a head-wearable device, and one of the cameras 3218 can be integrated with a lens assembly of the head-wearable device.

One or more of the electronic devices 3002 can include one or more haptic controllers 3012 and associated componentry for providing haptic events at one or more of the electronic devices 3002 (e.g., a vibrating sensation or audio output in response to an event at the electronic device 3002). The haptic controllers 3012 can communicate with one or more electroacoustic devices, including a speaker of the one or more speakers 3214 and/or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). The haptic controller 3012 can provide haptic events that are capable of being sensed by a user of the electronic devices 3002. In some embodiments, the one or more haptic controllers 3012 can receive input signals from an application of the applications 3430.

Memory 3400 optionally includes high-speed random-access memory and optionally also includes non-volatile memory, such as one or more magnetic disk storage devices, flash memory devices, or other non-volatile solid-state memory devices. Access to the memory 3400 by other components of the electronic device 3002, such as the one or more processors of the central processing unit 3004, and the peripherals interface 3014 is optionally controlled by a memory controller of the controllers 3010.

In some embodiments, software components stored in the memory 3400 can include one or more operating systems 3402 (e.g., a Linux-based operating system, an Android operating system, etc.). The memory 3400 can also include data 3410, including structured data (e.g., SQL databases, MongoDB databases, GraphQL data, JSON data, etc.). The data 3410 can include profile data 3412, sensor data 3414, media file data 3416, image storage 3418, and haptic-rendering algorithms 3420. In some embodiments, the haptic-rendering algorithms 3420 can include predetermined waveforms (or equations or methods used to determine waveform characteristics) to determine the haptic precursors needed to effectuate certain haptic sensations.

In some embodiments, software components stored in the memory 3400 include one or more applications 3430 configured to perform operations at the electronic devices 3002. In some embodiments, the one or more applications 3430 include one or more communication interface modules 3432, one or more graphics modules 3434, and/or one or more haptic-rendering application modules 3436. In some embodiments, a plurality of applications 3430 can work in conjunction with one another to perform various tasks at one or more of the electronic devices 3002. In some embodiments, the haptic-rendering application modules 3436 comprise software used to manage the repurposing or dual-purposing of electrodes for haptic-rendering functions.

It should be appreciated that the electronic devices 3002 are only some examples of the electronic devices 3002 within the computing system 3000, and that other electronic devices 3002 that are part of the computing system 3000 can have more or fewer components than shown optionally combining two or more components, or optionally having a different configuration or arrangement of the components. The various components shown in FIG. 3C are implemented in hardware, software, firmware, or a combination thereof, including one or more signal processing and/or application-specific integrated circuits.

As illustrated by the lower portion of FIG. 3C, various individual components of a wrist-wearable device can be examples of the electronic device 3002. For example, some or all of the components shown in the electronic device 3002 can be housed or otherwise disposed in a combined watch device 3002A, or within individual components of the capsule device watch body 3002B, the cradle portion 3002C, and/or a watch band.

FIG. 3D illustrates a wearable device 3170, in accordance with some embodiments. In some embodiments, the wearable device 3170 is used to generate control information (e.g., sensed data about neuromuscular signals or instructions to perform certain commands after the data is sensed) for causing a computing device to perform one or more input commands. In some embodiments, the wearable device 3170 includes a plurality of neuromuscular sensors 3176. In some embodiments, the plurality of neuromuscular sensors 3176 includes a predetermined number (e.g., 16) of neuromuscular sensors (e.g., EMG sensors) arranged circumferentially around an elastic band 3174. The plurality of neuromuscular sensors 3176 may include any suitable number of neuromuscular sensors. In some embodiments, the number and arrangement of neuromuscular sensors 3176 depends on the particular application for which the wearable device 3170 is used. For instance, a wearable device 3170 configured as an armband, wristband, or chest-band may include a plurality of neuromuscular sensors 3176 with different number of neuromuscular sensors and different arrangement for each use case, such as medical use cases as compared to gaming or general day-to-day use cases. For example, at least 16 neuromuscular sensors 3176 may be arranged circumferentially around elastic band 3174.

In some embodiments, the elastic band 3174 is configured to be worn around a user's lower arm or wrist. The elastic band 3174 may include a flexible electronic connector 3172. In some embodiments, the flexible electronic connector 3172 interconnects separate sensors and electronic circuitry that are enclosed in one or more sensor housings. Alternatively, in some embodiments, the flexible electronic connector 3172 interconnects separate sensors and electronic circuitry that are outside of the one or more sensor housings. Each neuromuscular sensor of the plurality of neuromuscular sensors 3176 can include a skin-contacting surface that includes one or more electrodes. One or more sensors of the plurality of neuromuscular sensors 3176 can be coupled together using flexible electronics incorporated into the wearable device 3170. In some embodiments, one or more sensors of the plurality of neuromuscular sensors 3176 can be integrated into a woven fabric, wherein one or more sensors of the plurality of neuromuscular sensors 3176 are sewn into the fabric and mimic the pliability of fabric (e.g., the one or more sensors of the plurality of neuromuscular sensors 3176 can be constructed from a series of woven strands of fabric). In some embodiments, the sensors are flush with the surface of the textile and are indistinguishable from the textile when worn by the user.

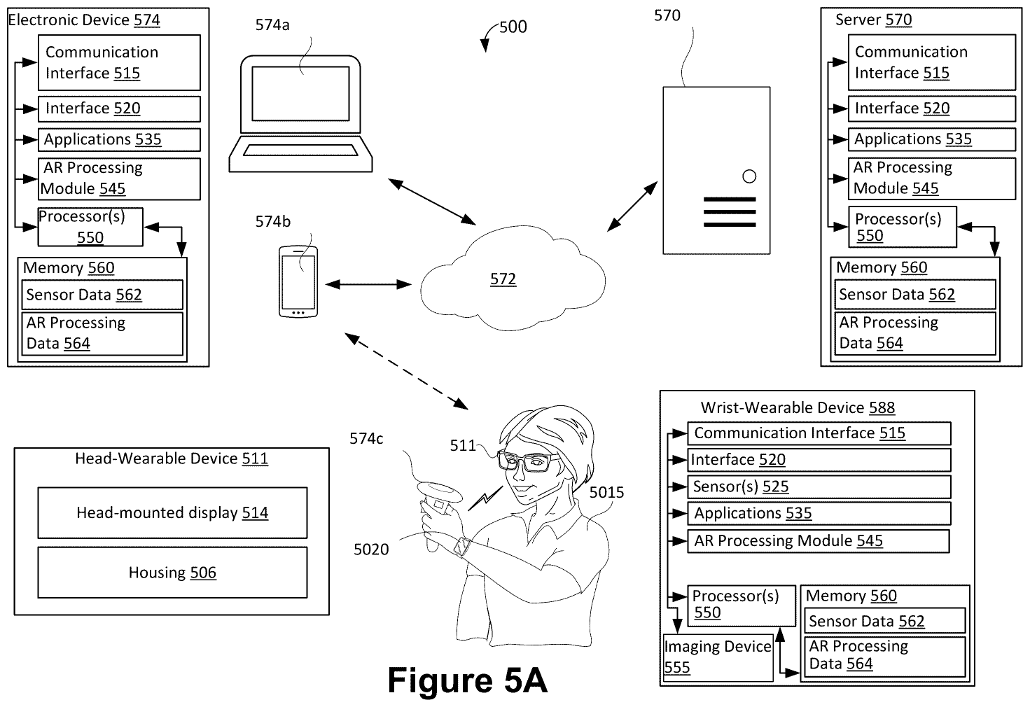

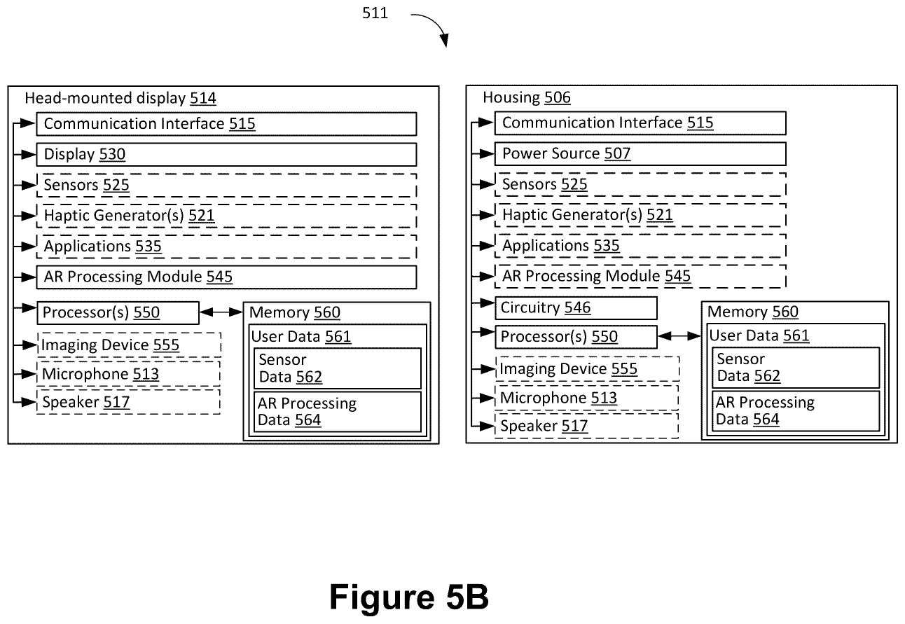

FIG. 3E illustrates a wearable device 3179 in accordance with some embodiments. The wearable device 3179 includes paired sensor channels 3185a-3185f along an interior surface of a wearable structure 3175 that are configured to detect neuromuscular signals. Different number of paired sensors channels can be used (e.g., one pair of sensors, three pairs of sensors, four pairs of sensors, or six pairs of sensors). The wearable structure 3175 can include a band portion 3190, a capsule portion 3195, and a cradle portion (not pictured) that is coupled with the band portion 3190 to allow for the capsule portion 3195 to be removably coupled with the band portion 3190. For embodiments in which the capsule portion 3195 is removable, the capsule portion 3195 can be referred to as a removable structure, such that in these embodiments the wearable device includes a wearable portion (e.g., band portion 3190 and the cradle portion) and a removable structure (the removable capsule portion that can be removed from the cradle). In some embodiments, the capsule portion 3195 includes the one or more processors and/or other components of the wearable device 588 described above in reference to FIG. 5. The wearable structure 3175 is configured to be worn by a user. More specifically, the wearable structure 3175 is configured to couple the wearable device 3179 to a wrist, arm, forearm, or other portion of the user's body. Each paired sensor channels 3185a-3185f includes two electrodes 3180 (e.g., electrodes 3180a-3180h) for sensing neuromuscular signals based on differential sensing within each respective sensor channel. In accordance with some embodiments, the wearable device 3170 further includes an electrical ground and a shielding electrode.

The techniques described above can be used with any device for sensing neuromuscular signals, including the arm-wearable devices of FIG. 3A-3C, but could also be used with other types of wearable devices for sensing neuromuscular signals (such as body-wearable or head-wearable devices that might have neuromuscular sensors closer to the brain or spinal column).

In some embodiments, a wrist-wearable device can be used in conjunction with a head-wearable device described below, and the wrist-wearable device can also be configured to be used to allow a user to control aspects of the artificial reality (e.g., by using EMG-based gestures to control user interface objects in the artificial reality and/or by allowing a user to interact with the touchscreen on the wrist-wearable device to also control aspects of the artificial reality). Having thus described example wrist-wearable device, attention will now be turned to example head-wearable devices, such AR glasses and VR headsets.

### Example Head-Wearable Devices

FIG. 4A shows an example AR system 400 in accordance with some embodiments. In FIG. 4A, the AR system 400 includes an eyewear device with a frame 402 configured to hold a left display device 406-1 and a right display device 406-2 in front of a user's eyes. The display devices 406-1 and 406-2 may act together or independently to present an image or series of images to a user. While the AR system 400 includes two displays, embodiments of this disclosure may be implemented in AR systems with a single near-eye display (NED) or more than two NEDs.

In some embodiments, the AR system 400 includes one or more sensors, such as the acoustic sensors 404. For example, the acoustic sensors 404 can generate measurement signals in response to motion of the AR system 400 and may be located on substantially any portion of the frame 402. Any one of the sensors may be a position sensor, an IMU, a depth camera assembly, or any combination thereof. In some embodiments, the AR system 400 includes more or fewer sensors than are shown in FIG. 4A. In embodiments in which the sensors include an IMU, the IMU may generate calibration data based on measurement signals from the sensors. Examples of the sensors include, without limitation, accelerometers, gyroscopes, magnetometers, other suitable types of sensors that detect motion, sensors used for error correction of the IMU, or some combination thereof.

In some embodiments, the AR system 400 includes a microphone array with a plurality of acoustic sensors 404-1 through 404-8, referred to collectively as the acoustic sensors 404. The acoustic sensors 404 may be transducers that detect air pressure variations induced by sound waves. In some embodiments, each acoustic sensor 404 is configured to detect sound and convert the detected sound into an electronic format (e.g., an analog or digital format). In some embodiments, the microphone array includes ten acoustic sensors: 404-1 and 404-2 designed to be placed inside a corresponding ear of the user, acoustic sensors 404-3, 404-4, 404-5, 404-6, 404-7, and 404-8 positioned at various locations on the frame 402, and acoustic sensors positioned on a corresponding neckband, where the neckband is an optional component of the system that is not present in certain embodiments of the artificial-reality systems discussed herein.

The configuration of the acoustic sensors 404 of the microphone array may vary. While the AR system 400 is shown in FIG. 4A having ten acoustic sensors 404, the number of acoustic sensors 404 may be more or fewer than ten. In some situations, using more acoustic sensors 404 increases the amount of audio information collected and/or the sensitivity and accuracy of the audio information. In contrast, in some situations, using a lower number of acoustic sensors 404 decreases the computing power required by a controller to process the collected audio information. In addition, the position of each acoustic sensor 404 of the microphone array may vary. For example, the position of an acoustic sensor 404 may include a defined position on the user, a defined coordinate on the frame 402, an orientation associated with each acoustic sensor, or some combination thereof.

The acoustic sensors 404-1 and 404-2 may be positioned on different parts of the user's ear. In some embodiments, there are additional acoustic sensors on or surrounding the ear in addition to acoustic sensors 404 inside the ear canal. In some situations, having an acoustic sensor positioned next to an ear canal of a user enables the microphone array to collect information on how sounds arrive at the ear canal. By positioning at least two of the acoustic sensors 404 on either side of a user's head (e.g., as binaural microphones), the AR device 400 is able to simulate binaural hearing and capture a three-dimensional (3D) stereo sound field around a user's head. In some embodiments, the acoustic sensors 404-1 and 404-2 are connected to the AR system 400 via a wired connection, and in other embodiments, the acoustic sensors 404-1 and 404-2 are connected to the AR system 400 via a wireless connection (e.g., a Bluetooth connection). In some embodiments, the AR system 400 does not include the acoustic sensors 404-1 and 404-2.

The acoustic sensors 404 on the frame 402 may be positioned along the length of the temples, across the bridge of the nose, above or below the display devices 406, or in some combination thereof. The acoustic sensors 404 may be oriented such that the microphone array is able to detect sounds in a wide range of directions surrounding the user who is wearing the AR system 400. In some embodiments, a calibration process is performed during manufacturing of the AR system 400 to determine relative positioning of each acoustic sensor 404 in the microphone array.

In some embodiments, the eyewear device further includes, or is communicatively coupled to, an external device (e.g., a paired device), such as the optional neckband discussed above. In some embodiments, the optional neckband is coupled to the eyewear device via one or more connectors. The connectors may be wired or wireless connectors and may include electrical and/or non-electrical (e.g., structural) components. In some embodiments, the eyewear device and the neckband operate independently without any wired or wireless connection between them. In some embodiments, the components of the eyewear device and the neckband are located on one or more additional peripheral devices paired with the eyewear device, the neckband, or some combination thereof. Furthermore, the neckband is intended to represent any suitable type or form of paired device. Thus, the following discussion of neckband may also apply to various other paired devices, such as smart watches, smartphones, wristbands, other wearable devices, hand-held controllers, tablet computers, or laptop computers.

In some situations, pairing external devices, such as the optional neckband, with the AR eyewear device enables the AR eyewear device to achieve the form factor of a pair of glasses while still providing sufficient battery and computation power for expanded capabilities. Some, or all, of the battery power, computational resources, and/or additional features of the AR system 400 may be provided by a paired device or shared between a paired device and an eyewear device, thus reducing the weight, heat profile, and form factor of the eyewear device overall while still retaining desired functionality. For example, the neckband may allow components that would otherwise be included on an eyewear device to be included in the neckband thereby shifting a weight load from a user's head to a user's shoulders. In some embodiments, the neckband has a larger surface area over which to diffuse and disperse heat to the ambient environment. Thus, the neckband may allow for greater battery and computation capacity than might otherwise have been possible on a stand-alone eyewear device. Because weight carried in the neckband may be less invasive to a user than weight carried in the eyewear device, a user may tolerate wearing a lighter eyewear device and carrying or wearing the paired device for greater lengths of time than the user would tolerate wearing a heavy, stand-alone eyewear device, thereby enabling an artificial-reality environment to be incorporated more fully into a user's day-to-day activities.

In some embodiments, the optional neckband is communicatively coupled with the eyewear device and/or to other devices. The other devices may provide certain functions (e.g., tracking, localizing, depth mapping, processing, storage, etc.) to the AR system 400. In some embodiments, the neckband includes a controller and a power source. In some embodiments, the acoustic sensors of the neckband are configured to detect sound and convert the detected sound into an electronic format (analog or digital).

The controller of the neckband processes information generated by the sensors on the neckband and/or the AR system 400. For example, the controller may process information from the acoustic sensors 404. For each detected sound, the controller may perform a direction of arrival (DOA) estimation to estimate a direction from which the detected sound arrived at the microphone array. As the microphone array detects sounds, the controller may populate an audio data set with the information. In embodiments in which the AR system 400 includes an IMU, the controller may compute all inertial and spatial calculations from the IMU located on the eyewear device. The connector may convey information between the eyewear device and the neckband and between the eyewear device and the controller. The information may be in the form of optical data, electrical data, wireless data, or any other transmittable data form. Moving the processing of information generated by the eyewear device to the neckband may reduce weight and heat in the eyewear device, making it more comfortable and safer for a user.

In some embodiments, the power source in the neckband provides power to the eyewear device and the neckband. The power source may include, without limitation, lithium-ion batteries, lithium-polymer batteries, primary lithium batteries, alkaline batteries, or any other form of power storage. In some embodiments, the power source is a wired power source.

As noted, some artificial-reality systems may, instead of blending an artificial reality with actual reality, substantially replace one or more of a user's sensory perceptions of the real world with a virtual experience. One example of this type of system is a head-worn display system, such as the VR system 450 in FIG. 4B, which mostly or completely covers a user's field of view.

FIG. 4B shows a VR system 450 (e.g., also referred to herein as VR headsets or VR headset) in accordance with some embodiments. The VR system 450 includes a head-mounted display (HMD) 452. The HMD 452 includes a front body 456 and a frame 454 (e.g., a strap or band) shaped to fit around a user's head. In some embodiments, the HMD 452 includes output audio transducers 458-1 and 458-2, as shown in FIG. 4B (e.g., transducers). In some embodiments, the front body 456 and/or the frame 454 includes one or more electronic elements, including one or more electronic displays, one or more IMUs, one or more tracking emitters or detectors, and/or any other suitable device or sensor for creating an artificial-reality experience.

Artificial-reality systems may include a variety of types of visual feedback mechanisms. For example, display devices in the AR system 400 and/or the VR system 450 may include one or more liquid-crystal displays (LCDs), light emitting diode (LED) displays, organic LED (OLED) displays, and/or any other suitable type of display screen. Artificial-reality systems may include a single display screen for both eyes or may provide a display screen for each eye, which may allow for additional flexibility for varifocal adjustments or for correcting a refractive error associated with the user's vision. Some artificial-reality systems also include optical subsystems having one or more lenses (e.g., conventional concave or convex lenses, Fresnel lenses, or adjustable liquid lenses) through which a user may view a display screen.

In addition to or instead of using display screens, some artificial-reality systems include one or more projection systems. For example, display devices in the AR system 400 and/or the VR system 450 may include micro-LED projectors that project light (e.g., using a waveguide) into display devices, such as clear combiner lenses that allow ambient light to pass through. The display devices may refract the projected light toward a user's pupil and may enable a user to simultaneously view both artificial-reality content and the real world. Artificial-reality systems may also be configured with any other suitable type or form of image projection system.

Artificial-reality systems may also include various types of computer vision components and subsystems. For example, the AR system 400 and/or the VR system 450 can include one or more optical sensors such as two-dimensional (2D) or 3D cameras, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions. For example, FIG. 4B shows VR system 450 having cameras 460-1 and 460-2 that can be used to provide depth information for creating a voxel field and a two-dimensional mesh to provide object information to the user to avoid collisions. FIG. 4B also shows that the VR system includes one or more additional cameras 4662 that are configured to augment the cameras 460-1 and 460-2 by providing more information. For example, the additional cameras 4662 can be used to supply color information that is not discerned by cameras 460-1 and 460-2. In some embodiments, cameras 460-1 and 460-2 and additional cameras 4662 can include an optional IR cut filter configured to remove IR light from being received at the respective camera sensors.

In some embodiments, the AR system 400 and/or the VR system 450 can include haptic (tactile) feedback systems, which may be incorporated into headwear, gloves, body suits, handheld controllers, environmental devices (e.g., chairs or floormats), and/or any other type of device or system, such as the wearable devices discussed herein. The haptic feedback systems may provide various types of cutaneous feedback, including vibration, force, traction, shear, texture, and/or temperature. The haptic feedback systems may also provide various types of kinesthetic feedback, such as motion and compliance. The haptic feedback may be implemented using motors, piezoelectric actuators, fluidic systems, and/or a variety of other types of feedback mechanisms. The haptic feedback systems may be implemented independently of other artificial-reality devices, within other artificial-reality devices, and/or in conjunction with other artificial-reality devices.

The techniques described above can be used with any device for interacting with an artificial-reality environment, including the head-wearable devices of FIG. 4A-4B, but could also be used with other types of wearable devices for sensing neuromuscular signals (such as body-wearable or head-wearable devices that might have neuromuscular sensors closer to the brain or spinal column). Having thus described example wrist-wearable device and head-wearable devices, attention will now be turned to example feedback systems that can be integrated into the devices described above or be a separate device.

### Example Systems

FIG. 5A is a block diagram illustrating an example artificial-reality system in accordance with some embodiments. The system 500 includes one or more devices for facilitating an interactivity with an artificial-reality environment in accordance with some embodiments. For example, the head-wearable device 511 can present to the user 5015 with a user interface within the artificial-reality environment. As a non-limiting example, the system 500 includes one or more wearable devices, which can be used in conjunction with one or more computing devices. In some embodiments, the system 500 provides the functionality of a virtual-reality device, an augmented-reality device, a mixed-reality device, hybrid-reality device, or a combination thereof. In some embodiments, the system 500 provides the functionality of a user interface and/or one or more user applications (e.g., games, word processors, messaging applications, calendars, clocks, etc.).

The system 500 can include one or more of servers 570, electronic devices 574 (e.g., a computer 574a, a smartphone 574b, a controller 574c, and/or other devices), head-wearable devices 511 (e.g., the AR system 400 or the VR system 450), and/or wrist-wearable devices 588 (e.g., the wrist-wearable device 5020). In some embodiments, the one or more of servers 570, electronic devices 574, head-wearable devices 511, and/or wrist-wearable devices 588 are communicatively coupled via a network 572. In some embodiments, the head-wearable device 511 is configured to cause one or more operations to be performed by a communicatively coupled wrist-wearable device 588, and/or the two devices can also both be connected to an intermediary device, such as a smartphone 574b, a controller 574c, or other device that provides instructions and data to and between the two devices. In some embodiments, the head-wearable device 511 is configured to cause one or more operations to be performed by multiple devices in conjunction with the wrist-wearable device 588. In some embodiments, instructions to cause the performance of one or more operations are controlled via an artificial-reality processing module 545. The artificial-reality processing module 545 can be implemented in one or more devices, such as the one or more of servers 570, electronic devices 574, head-wearable devices 511, and/or wrist-wearable devices 588. In some embodiments, the one or more devices perform operations of the artificial-reality processing module 545, using one or more respective processors, individually or in conjunction with at least one other device as described herein. In some embodiments, the system 500 includes other wearable devices not shown in FIG. 5, such as rings, collars, anklets, gloves, and the like.

In some embodiments, the system 500 provides the functionality to control or provide commands to the one or more computing devices 574 based on a wearable device (e.g., head-wearable device 511 or wrist-wearable device 588) determining motor actions or intended motor actions of the user. A motor action is an intended motor action when before the user performs the motor action or before the user completes the motor action, the detected neuromuscular signals travelling through the neuromuscular pathways can be determined to be the motor action. Motor actions can be detected based on the detected neuromuscular signals, but can additionally (using a fusion of the various sensor inputs), or alternatively, be detected using other types of sensors (such as cameras focused on viewing hand movements and/or using data from an inertial measurement unit that can detect characteristic vibration sequences or other data types to correspond to particular in-air hand gestures). The one or more computing devices include one or more of a head-mounted display, smartphones, tablets, smart watches, laptops, computer systems, augmented reality systems, robots, vehicles, virtual avatars, user interfaces, a wrist-wearable device, and/or other electronic devices and/or control interfaces.

In some embodiments, the motor actions include digit movements, hand

movements, wrist movements, arm movements, pinch gestures, index finger movements, middle finger movements, ring finger movements, little finger movements, thumb movements, hand clenches (or fists), waving motions, and/or other movements of the user's hand or arm.

In some embodiments, the user can define one or more gestures using the learning module. In some embodiments, the user can enter a training phase in which a user-defined gesture is associated with one or more input commands that when provided to a computing device cause the computing device to perform an action. Similarly, the one or more input commands associated with the user-defined gesture can be used to cause a wearable device to perform one or more actions locally. The user-defined gesture, once trained, is stored in the memory 560. Similar to the motor actions, the one or more processors 550 can use the detected neuromuscular signals by the one or more sensors 525 to determine that a user-defined gesture was performed by the user.

The electronic devices 574 can also include a communication interface 515, an interface 520 (e.g., including one or more displays, lights, speakers, and haptic generators), one or more sensors 525, one or more applications 535, an artificial-reality processing module 545, one or more processors 550, and memory 560. The electronic devices 574 are configured to communicatively couple with the wrist-wearable device 588 and/or head-wearable device 511 (or other devices) using the communication interface 515. In some embodiments, the electronic devices 574 are configured to communicatively couple with the wrist-wearable device 588 and/or head-wearable device 511 (or other devices) via an API. In some embodiments, the electronic devices 574 operate in conjunction with the wrist-wearable device 588 and/or the head-wearable device 511 to determine a hand gesture and cause the performance of an operation or action at a communicatively coupled device.

The server 570 includes a communication interface 515, one or more applications 535, an artificial-reality processing module 545, one or more processors 550, and memory 560. In some embodiments, the server 570 is configured to receive sensor data from one or more devices, such as the head-wearable device 511, the wrist-wearable device 588, and/or electronic device 574, and use the received sensor data to identify a gesture or user input. The server 570 can generate instructions that cause the performance of operations and actions associated with a determined gesture or user input at communicatively coupled devices, such as the head-wearable device 511.

The head-wearable device 511 includes smart glasses (e.g., the augmented-reality glasses), artificial-reality headsets (e.g., VR/AR headsets), or other head-worn device. In some embodiments, one or more components of the head-wearable device 511 are housed within a body of the HMD 514 (e.g., frames of smart glasses, a body of an AR headset, etc.). In some embodiments, one or more components of the head-wearable device 511 are stored within or coupled with lenses of the HMD 514. Alternatively or in addition, in some embodiments, one or more components of the head-wearable device 511 are housed within a modular housing 506. The head-wearable device 511 is configured to communicatively couple with other electronic device 574 and/or a server 570 using communication interface 515 as discussed above.

FIG. 5B describes additional details of the HMD 514 and modular housing 506 described above in reference to FIG. 5A, in accordance with some embodiments.

The housing 506 include(s) a communication interface 515, circuitry 546, a power source 507 (e.g., a battery for powering one or more electronic components of the housing 506 and/or providing usable power to the HMD 514), one or more processors 550, and memory 560. In some embodiments, the housing 506 can include one or more supplemental components that add to the functionality of the HMD 514. For example, in some embodiments, the housing 506 can include one or more sensors 525, an AR processing module 545, one or more haptic generators 521, one or more imaging devices 555, one or more microphones 513, one or more speakers 517, etc. The housing 506 is configured to couple with the HMD 514 via the one or more retractable side straps. More specifically, the housing 506 is a modular portion of the head-wearable device 511 that can be removed from head-wearable device 511 and replaced with another housing (which includes more or less functionality). The modularity of the housing 506 allows a user to adjust the functionality of the head-wearable device 511 based on their needs.

In some embodiments, the communications interface 515 is configured to communicatively couple the housing 506 with the HMD 514, the server 570, and/or other electronic device 574 (e.g., a wrist-wearable device 5020, a tablet 574b, a computer 574a, etc.). The communication interface 515 is used to establish wired or wireless connections between the housing 506 and the other devices. In some embodiments, the communication interface 515 includes hardware capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol. In some embodiments, the housing 506 is configured to communicatively couple with the HMD 514 and/or other electronic device 574 via an API.

In some embodiments, the power source 507 is a battery. The power source 507 can be a primary or secondary battery source for the HMD 514. In some embodiments, the power source 507 provides useable power to the one or more electrical components of the housing 506 or the HMD 514. For example, the power source 507 can provide usable power to the sensors 525, the speakers 517, the HMD 514, and the microphone 513. In some embodiments, the power source 507 is a rechargeable battery. In some embodiments, the power source 507 is a modular battery that can be removed and replaced with a fully charged battery while it is charged separately.

The one or more sensors 525 can include heart rate sensors, neuromuscular-signal sensors (e.g., EMG sensors), SpO2 sensors, altimeters, thermal sensors or thermal couples, ambient light sensors, ambient noise sensors, and/or IMUs. Additional non-limiting examples of the one or more sensors 525 include, e.g., infrared, pyroelectric, ultrasonic, microphone, laser, optical, Doppler, gyro, accelerometer, resonant LC sensors, capacitive sensors, acoustic sensors, and/or inductive sensors. In some embodiments, the one or more sensors 525 are configured to gather additional data about the user (e.g., an impedance of the user's body). Examples of sensor data output by these sensors includes body temperature data, infrared range-finder data, positional information, motion data, activity recognition data, silhouette detection and recognition data, gesture data, heart rate data, and other wearable device data (e.g., biometric readings and output, accelerometer data). The one or more sensors 525 can include location-sensing devices (e.g., GPS) configured to provide location information. In some embodiments, the data measured or sensed by the one or more sensors 525 is stored in memory 560. In some embodiments, the housing 506 receives sensor data from communicatively coupled devices, such as the HMD 514, the server 570, and/or other electronic device 574. Alternatively, the housing 506 can provide sensor data to the HMD 514, the server 570, and/or other electronic device 574.

The one or more haptic generators 521 can include one or more actuators (e.g., eccentric rotating mass (ERM), linear resonant actuators (LRA), voice coil motor (VCM), piezo haptic actuator, thermoelectric devices, solenoid actuators, ultrasonic transducers or sensors, etc.). In some embodiments, the one or more haptic generators 521 are hydraulic, pneumatic, electric, and/or mechanical actuators. In some embodiments, the one or more haptic generators 521 are part of a surface of the housing 506 that can be used to generate a haptic response (e.g., a thermal change at the surface, a tightening or loosening of a band, increase or decrease in pressure, etc.). For example, the one or more haptic generators 521 can apply vibration stimulations, pressure stimulations, squeeze simulations, shear stimulations, temperature changes, or some combination thereof to the user. In addition, in some embodiments, the one or more haptic generators 521 include audio generating devices (e.g., speakers 517 and other sound transducers) and illuminating devices (e.g., light-emitting diodes (LED) s, screen displays, etc.). The one or more haptic generators 521 can be used to generate different audible sounds and/or visible lights that are provided to the user as haptic responses. The above list of haptic generators is non-exhaustive; any affective devices can be used to generate one or more haptic responses that are delivered to a user.

In some embodiments, the one or more applications 535 include social-media applications, banking applications, health applications, messaging applications, web browsers, gaming applications, streaming applications, media applications, imaging applications, productivity applications, social applications, etc. In some embodiments, the one or more applications 535 include artificial-reality applications. The one or more applications 535 are configured to provide data to the head-wearable device 511 for performing one or more operations. In some embodiments, the one or more applications 535 can be displayed via a display 530 of the head-wearable device 511 (e.g., via the HMD 514).

In some embodiments, instructions to cause the performance of one or more operations are controlled via an AR processing module 545. The AR processing module 545 can be implemented in one or more devices, such as the one or more of servers 570, electronic devices 574, head-wearable devices 511, and/or wrist-wearable devices. In some embodiments, the one or more devices perform operations of the AR processing module 545, using one or more respective processors, individually or in conjunction with at least one other device as described herein. In some embodiments, the AR processing module 545 is configured process signals based at least on sensor data. In some embodiments, the AR processing module 545 is configured process signals based on image data received that captures at least a portion of the user's hand, mouth, facial expression, surroundings, etc. For example, the housing 506 can receive EMG data and/or IMU data from one or more sensors 525 and provide the sensor data to the AR processing module 545 for a particular operation (e.g., gesture recognition, facial recognition, etc.). The AR processing module 545 causes a device communicatively coupled to the housing 506 to perform an operation (or action). In some embodiments, the AR processing module 545 performs different operations based on the sensor data and/or performs one or more actions based on the sensor data.

In some embodiments, the one or more imaging devices 555 can include an ultra-wide camera, a wide camera, a telephoto camera, a depth-sensing camera, or other types of cameras. In some embodiments, the one or more imaging devices 555 are used to capture image data and/or video data. The imaging devices 555 can be coupled to a portion of the housing 506. The captured image data can be processed and stored in memory and then presented to a user for viewing. The one or more imaging devices 555 can include one or more modes for capturing image data or video data. For example, these modes can include a high-dynamic range (HDR) image capture mode, a low-light image capture mode, burst-image capture mode, and other modes. In some embodiments, a particular mode is automatically selected based on the environment (e.g., lighting, movement of the device, etc.). For example, a wrist-wearable device with HDR image capture mode and a low-light image capture mode active can automatically select the appropriate mode based on the environment (e.g., dark lighting may result in the use of low-light image capture mode instead of HDR image capture mode). In some embodiments, the user can select the mode. The image data and/or video data captured by the one or more imaging devices 555 is stored in memory 560 (which can include volatile and non-volatile memory such that the image data and/or video data can be temporarily or permanently stored, as needed, depending on the circumstances).

The circuitry 546 is configured to facilitate the interaction between the housing 506 and the HMD 514. In some embodiments, the circuitry 546 is configured to regulate the distribution of power between the power source 507 and the HMD 514. In some embodiments, the circuitry 546 is configured to transfer audio and/or video data between the HMD 514 and/or one or more components of the housing 506.

The one or more processors 550 can be implemented as any kind of computing device, such as an integrated system-on-a-chip, a microcontroller, a fixed programmable gate array (FPGA), a microprocessor, and/or other application-specific integrated circuits (ASICs). The processor may operate in conjunction with memory 560. The memory 560 may be or include random access memory (RAM), read-only memory (ROM), dynamic random access memory (DRAM), static random access memory (SRAM) and magnetoresistive random access memory (MRAM), and may include firmware, such as static data or fixed instructions, basic input/output system (BIOS), system functions, configuration data, and other routines used during the operation of the housing and the processor 550. The memory 560 also provides a storage area for data and instructions associated with applications and data handled by the processor 550.

In some embodiments, the memory 560 stores at least user data 561 including sensor data 562 and AR processing data 564. The sensor data 562 includes sensor data monitored by one or more sensors 525 of the housing 506 and/or sensor data received from one or more devices communicatively coupled with the housing 506, such as the HMD 514, the smartphone 574b, the wrist-wearable device 5020, etc. The sensor data 562 can include sensor data collected over a predetermined period of time that can be used by the AR processing module 545. The AR processing data 564 can include one or more predefined camera-controlled gestures, user-defined camera-control gestures, predefined non-camera-control gestures, and/or user defined non-camera-control gestures. In some embodiments, the AR processing data 564 further includes one or more predetermined thresholds for different gestures.

The HMD 514 includes a communication interface 515, a display 530, an AR processing module 545, one or more processors, and memory. In some embodiments, the HMD 514 includes one or more sensors 525, one or more haptic generators 521, one or more imaging devices 555 (e.g., a camera), microphones 513, speakers 517, and/or one or more applications 535. The HMD 514 operates in conjunction with the housing 506 to perform one or more operations of a head-wearable device 511, such as capturing camera data, presenting a representation of the image data at a coupled display, operating one or more applications 535, and/or allowing a user to participate in an AR environment.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements; these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A method of repurposing a sensing component to also perform a haptic-rendering function, the method comprising:
at a first point in time, using a sensing component of a wearable electronic device to sense a biometric signal of a user; and
at a second point in time that is distinct from the first point in time, instructing the sensing component to send a predetermined haptic precursor to a targeted biophysical area of the user, such that the user is caused to perceive a haptic sensation after the predetermined haptic precursor is received at the targeted biophysical area of the user.

2. The method of claim 1, wherein the sensing component is an electrode configured to sense one or more neuromuscular signals, and the predetermined haptic precursor is an electrical current sent to the targeted biophysical area of the user.

3. The method of claim 1, including:
at a third point in time that is distinct from the first point in time and the second point in time, instructing the sensing component to send another predetermined haptic precursor to the targeted biophysical area of the user, wherein the other predetermined haptic precursor is different from the haptic precursor, such that the user is caused to perceive another haptic sensation after the haptic precursor is received at the targeted biophysical area of the user.

4. The method of claim 1, wherein the haptic sensation is one or more of a muscular movement, a nerve stimulation, a tendon activation, and a skin-feedback sensation.

5. The method of claim 1, including:
at a third point in time, instructing the sensing component to send a predetermined haptic precursor to another targeted biophysical area of the user different than the targeted biophysical area, such that the user is caused to perceive a different haptic sensation after the haptic precursor is received at the other targeted biophysical area of the user.

6. The method of claim 1, including:
at a third point in time, using another sensing component to sense another biometric signal of a user; and
at a fourth point in time, instructing the other sensing component to send another predetermined haptic precursor to another targeted biophysical area of the user, such that the user is caused to perceive another haptic sensation after the other haptic precursor is received at the targeted biophysical area of the user.

7. The method of claim 1, including:
at a second point in time that is distinct from the first point in time, instructing both the sensing component and another sensing component to send another predetermined haptic precursor to the targeted biophysical area of the user, such that the user is caused to perceive the haptic sensation after the haptic precursor is received at the targeted biophysical area of the user.

8. The method of claim 1, including:
at a second point in time that is distinct from the first point in time, instructing:
the sensing component to send the predetermined haptic precursor to the targeted biophysical area of the user, such that the user is caused to perceive the haptic sensation after the haptic precursor is received at the targeted biophysical area of the user; and
another sensing component to send another haptic precursor to another targeted biophysical area of the user, such that the user is caused to perceive another haptic sensation after the other haptic precursor is received at the other targeted biophysical area of the user.

9. The method of claim 1, wherein the user is caused to perceive the haptic sensation without the user wearing a glove.

10. The method of claim 1, wherein:
the wearable electronic device is a wrist-wearable device that is configured to be in communication with a head-wearable device, and
the haptic sensation is configured to be provided when a hand of the user is determined to be within a predetermined distance of an object presented to the user via the head-wearable device.

11. A system including a wearable device and a head-wearable display device, the system configured to present user interfaces via the head-wearable device, and the wearable device configured to:
at a first point in time, use a sensing component of a wearable electronic device to sense a biometric signal of a user; and
at a second point in time that is distinct from the first point in time, instruct the sensing component to send a predetermined haptic precursor to a targeted biophysical area of the user, such that the user is caused to perceive a haptic sensation after the predetermined haptic precursor is received at the targeted biophysical area of the user.

12. The system of claim 11, wherein the sensing component is an electrode configured to sense one or more neuromuscular signals, and the predetermined haptic precursor is an electrical current sent to the targeted biophysical area of the user.

13. The system of claim 11, wherein the system is further configured to:
at a third point in time that is distinct from the first point in time and the second point in time, instruct the sensing component to send another predetermined haptic precursor to the targeted biophysical area of the user, wherein the other predetermined haptic precursor is different from the haptic precursor, such that the user is caused to perceive another haptic sensation after the haptic precursor is received at the targeted biophysical area of the user.

14. The system of claim 11, wherein the haptic sensation is one or more of a muscular movement, a nerve stimulation, a tendon activation, and a skin-feedback sensation.

15. The system of claim 11, wherein the system is further configured to:
at a third point in time, use another sensing component to sense another biometric signal of a user; and
at a fourth point in time, instruct the other sensing component to send another predetermined haptic precursor to another targeted biophysical area of the user, such that the user is caused to perceive another haptic sensation after the other haptic precursor is received at the targeted biophysical area of the user.

16. A non-transitory computer-readable storage medium including instructions that, when executed by a wrist-wearable device, cause the wrist-wearable device to: at a first point in time, use a sensing component of a wearable electronic device to sense a biometric signal of a user; and
at a second point in time that is distinct from the first point in time, instruct the sensing component to send a predetermined haptic precursor to a targeted biophysical area of the user, such that the user is caused to perceive a haptic sensation after the predetermined haptic precursor is received at the targeted biophysical area of the user.

17. The non-transitory computer-readable-storage medium of claim 16, wherein the sensing component is an electrode configured to sense one or more neuromuscular signals, and the predetermined haptic precursor is an electrical current sent to the targeted biophysical area of the user.

18. The non-transitory computer-readable-storage medium of claim 16, wherein the instructions that, when executed by a wrist-wearable device, further cause the wrist-wearable device to:
at a third point in time that is distinct from the first point in time and the second point in time, instruct the sensing component to send another predetermined haptic precursor to the targeted biophysical area of the user, wherein the other predetermined haptic precursor is different from the haptic precursor, such that the user is caused to perceive another haptic sensation after the haptic precursor is received at the targeted biophysical area of the user.

19. The non-transitory computer-readable-storage medium of claim 16, wherein the haptic sensation is one or more of a muscular movement, a nerve stimulation, a tendon activation, and a skin-feedback sensation.

20. The non-transitory computer-readable-storage medium of claim 16, wherein the instructions that, when executed by a wrist-wearable device, further cause the wrist-wearable device to:
at a third point in time, use another sensing component to sense another biometric signal of a user; and
at a fourth point in time, instruct the other sensing component to send another predetermined haptic precursor to another targeted biophysical area of the user, such that the user is caused to perceive another haptic sensation after the other haptic precursor is received at the targeted biophysical area of the user.
